(19) United States
(12) Patent Application Publication
(10) Pub. No.: US 2023/0182283 A1
(43) Pub. Date: Jun. 15, 2023

(54) CONTINUUM ROBOT

(71) Applicant: CANON KABUSHIKI KAISHA, Tokyo (JP)

(72) Inventors: DAISUKE KANEKO, Kanagawa (JP); YUSUKE NIIKAWA, Kanagawa (JP); YASUMICHI ARIMITSU, Kanagawa (JP); ISAO MATSUOKA, Kanagawa (JP)

(21) Appl. No.: 18/060,925

(22) Filed: Dec. 1, 2022

(30) Foreign Application Priority Data
Dec. 14, 2021 (JP) 2021-202426

(51) Int. Cl. B25J 9/00; B25J 9/12

(52) U.S. Cl. CPC B25J 9/0015; B25J 9/123

(57) ABSTRACT

One or more embodiments of a continuum robot may include a bendable body having linear members provided along a first pitch circle, and motors respectively having output shafts provided along a second pitch circle offset outside from the first pitch circle and being configured to respectively drive linear members to bend the bendable body; intermediate supporting shafts provided along a third pitch circle offset outside from the first pitch circle and offset inside from the second pitch circle; first connection members respectively connecting end portions of the linear members and the intermediate supporting shafts to each other; and second connection members respectively connecting the intermediate supporting shafts and the output shafts to each other and configured to convert rotation of each of the output shafts into rectilinear motion to cause each of the intermediate supporting shafts to rectilinearly move.

### BACKGROUND OF THE INVENTION

### Field of the Invention

Aspects of the present disclosure generally relate to one or more embodiments of a continuum robot.

### Description of the Related Art

Japanese Unexamined Patent Application Publication (Translation of PCT Application) No. 2020-518353 discusses a continuum robot including a bendable body, which has driving wires, and actuators and being configured to cause the actuators to retract and advance the driving wires, thus maneuvering the bendable body.

In a continuum robot which is moved by driving a plurality of wires disposed along the pitch circle, a plurality of motors is required as drive sources for the wires.

In this case, as discussed in Japanese Unexamined Patent Application Publication No. 2020-518353, there is conceivable a configuration in which, to avoid interferences between the motors, a motor shaft is arranged at a position which is offset outside in the radial direction of the pitch circle with respect to the end portion of the wire and the end portion of the wire is connected to the motor shaft via a traction unit or a tractor.

However, in such a configuration, as the distance between the end portion of the wire and the motor shaft becomes larger, a moment which is applied to the tractor during driving of the wire becomes larger. Therefore, abrasion occurring between the motor shaft and the tractor becomes larger, such that endurance may deteriorate and the wire may be blocked from smoothly moving.

Moreover, if, to make the distance between the end portion of the wire and the motor shaft smaller, the wire is configured to be guided in such a way as to be bent to the outside in the radial direction of the pitch circle, a frictional force in a direction to block the movement of the wire occurs during driving of the wire. Therefore, a loss occurs in driving force transmission to the wire, such that the wire may be blocked from smoothly moving.

### SUMMARY OF THE INVENTION

Aspects of the present disclosure are generally directed to providing a continuum robot capable of improving endurance and implementing a smooth movement of a linear member.

According to at least one aspect of the present disclosure, at least one embodiment of a continuum robot may include: a bendable body having a plurality of linear members provided along a first pitch circle, and a plurality of motors respectively having a plurality of output shafts provided along a second pitch circle which is offset outside from the first pitch circle and being configured to respectively drive the plurality of linear members to bend the bendable body; a plurality of intermediate supporting shafts provided along a third pitch circle which is offset outside from the first pitch circle and is offset inside from the second pitch circle; a plurality of first connection members respectively connecting end portions of the plurality of linear members and the plurality of intermediate supporting shafts to each other; and a plurality of second connection members respectively connecting the plurality of intermediate supporting shafts and the plurality of output shafts to each other and configured to convert rotation of each of the plurality of output shafts into rectilinear motion to cause each of the plurality of intermediate supporting shafts to rectilinearly move.

According to other aspects of the present disclosure, one or more additional continuum robots and one or more methods are discussed herein. Further features of the present disclosure will become apparent from the following description of exemplary embodiments with reference to the attached drawings.

### BRIEF DESCRIPTION OF THE DRAWINGS

FIG. 1 is an overall view of a medical system according to at least one embodiment of the present disclosure.

FIG. 2 is a perspective view illustrating a medical device and a support base according to at least one embodiment of the present disclosure.

FIGS. 3A and 3B are explanatory diagrams of a catheter according to at least one embodiment of the present disclosure.

FIGS. 4A and 4B are explanatory diagrams of a catheter unit or portion according to at least one embodiment of the present disclosure.

FIGS. 5A, 5B, and 5C are explanatory diagrams of a base unit or portion and a wire driving unit or portion according to at least one embodiment of the present disclosure.

FIGS. 6A, 6B, and 6C are explanatory diagrams of the wire driving unit or portion, a coupling device, and a bending driving portion according to at least one embodiment of the present disclosure.

FIGS. 7A and 7B are explanatory diagrams of mounting of the catheter unit or portion according to at least one embodiment of the present disclosure.

FIGS. 8A and 8B are diagrams used to explain coupling between the catheter unit or portion and the base unit or portion according to at least one embodiment of the present disclosure.

FIG. 9 is an exploded view used to explain coupling between the catheter unit or portion and the base unit or portion according to at least one embodiment of the present disclosure.

FIG. 10 is a diagram used to explain fixing of a driving wire by a coupling portion according to at least one embodiment of the present disclosure.

FIG. 11 is a diagram used to explain fixing of the driving wire by the coupling portion according to at least one embodiment of the present disclosure.

FIG. 12 is a diagram used to explain fixing of the driving wire by the coupling portion according to at least one embodiment of the present disclosure.

FIG. 13 is a diagram used to explain fixing of the driving wire by the coupling portion according to at least one embodiment of the present disclosure.

FIG. 14 is a diagram used to explain fixing of the driving wire by the coupling portion according to at least one embodiment of the present disclosure.

FIGS. 15A, 15B, and 15C are explanatory diagrams of the catheter unit or portion and the base unit or portion according to at least one embodiment of the present disclosure.

FIGS. 16A, 16B, and 16C are diagrams used to explain operations of an operation unit or portion according to at least one embodiment of the present disclosure.

FIGS. 17A, 17B, and 17C are diagrams used to explain operations of the operation unit or portion according to at least one embodiment of the present disclosure.

FIG. 18 is a sectional view used to explain a layout of a driving force transmission mechanism according to at least one embodiment of the present disclosure.

FIGS. 19A and 19B are sectional enlarged views used to explain the layout of the driving force transmission mechanism according to at least one embodiment of the present disclosure.

### DESCRIPTION OF THE EMBODIMENTS

Various exemplary embodiments, features, and aspects of the present disclosure will be described in detail below with reference to the drawings. Furthermore, for example, the dimension, material, shape, and location of each constituent component described in the following exemplary embodiments may be modified or altered as appropriate depending on, for example, configurations of one or more embodiments of devices to which one or more features of the present disclosure are applied and various conditions.

### Medical System and Medical Device Embodiment Examples

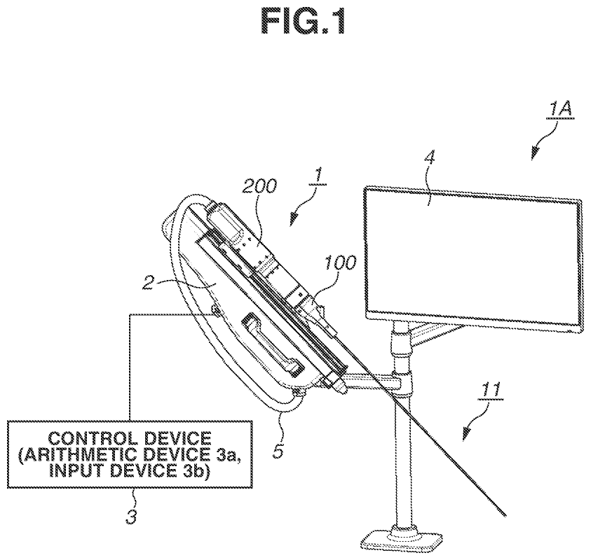

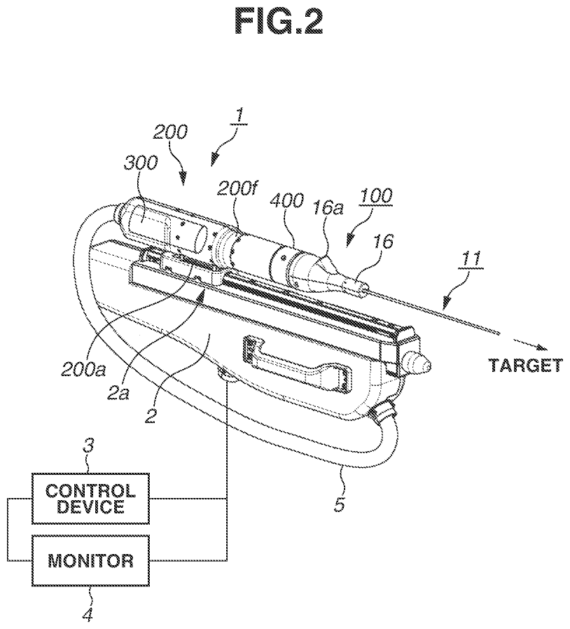

At least one embodiment of a medical system 1A and a medical device (a continuum robot) 1 are described with reference to FIG. 1 and FIG. 2. FIG. 1 is an overall view of the medical system 1A according to at least one embodiment of the present disclosure. FIG. 2 is a perspective view illustrating the medical device 1 and a support base 2 according to at least one embodiment of the present disclosure.

The medical system 1A includes the medical device 1, the support base 2 (to which the medical device 1 is attached), and a control device 3, which controls the medical device 1. In at least one embodiment, the medical system 1A is equipped with a monitor 4, which serves as a display device.

The medical device 1 includes a catheter unit (a bendable unit) or portion 100, which includes a catheter 11 serving as a bendable body, and a base unit (a driving unit or a mounted unit) or portion 200. The catheter unit or portion 100 is configured to be mountable to and detachable from the base unit or portion 200.

In the present exemplary embodiment, the user of the medical system 1A and the medical device 1 is able to insert the catheter 11 into a target to perform operations such as observation of the inside of the target, extraction of various samples from the inside of the target, and treatment for the inside of the target. In one exemplary embodiment, the user is able to insert the catheter 11 into a patient serving as a target. By way of at least one example, the user may insert the catheter 11 into the bronchi via the oral cavity or nasal cavity of a patient to perform operations such as observation, extraction, and excision of lung tissue.

The catheter 11 is able to be used, or operates, as a guide (sheath) which guides a medical tool for performing one or more of the above-mentioned operations. Examples of the medical tool (tool) include, but are not limited to, an endoscope, forceps, and an ablation device. Moreover, the catheter 11 itself may have a function or functions of, and may operate as, the above-mentioned medical tool.

In at least one embodiment, the control device 3 includes an arithmetic device 3a and an input device 3b. The input device 3b receives an instruction or input for maneuvering the catheter 11. The arithmetic device 3a may include: a storage which stores one or more programs and various pieces of data for controlling a catheter; a random access memory; and a central processing unit or processor that operates to execute the one or more programs. Moreover, the control device 3 may include an output unit or processor, which outputs a signal for displaying an image on the monitor 4.

As illustrated in FIG. 2, in at least one embodiment, the medical device 1 is electrically connected to the support base 2 and to control device 3 via a cable 5, which interconnects the base unit or portion 200 of the medical device 1 and the support base 2. Furthermore, the medical device 1 and the control device 3 may be directly connected to each other via a cable. The medical device 1 and the control device 3 may be connected to each other wirelessly.

The medical device 1 is detachably mounted to the support base 2 via the base unit or portion 200 in one or more embodiments.

More specifically, in at least one embodiment of the medical device 1, an attachment portion 200a of the base unit or portion 200 is detachably mounted to a moving stage (a receiving portion) 2a of the support base 2. The connection between the medical device 1 and the control device 3 is maintained in such a manner that the medical device 1 is able to be controlled by the control device 3 even in a state in which the attachment portion 200a of the medical device 1 has been detached from the moving stage 2a. In at least one embodiment, even in a state in which the attachment portion 200a of the medical device 1 has been detached from the moving stage 2a, the medical device 1 and the support base 2 are connected to each other via the cable 5.

The user may manually move the medical device 1 in a state in which the medical device 1 has been detached from the support base 2 (a state in which the medical device 1 has been detached from the moving stage 2a), thus being able to insert the catheter 11 into a target.

The user is able to use the medical device 1 in a state in which the catheter 11 has been inserted into a target and the medical device 1 has been attached to the support base 2. Specifically, in response to the moving stage 2a moving in a state in which the medical device 1 has been attached to the moving stage 2a, the medical device 1 moves. Then, an operation in which the medical device 1 moves in a direction to insert the catheter 11 into a target and an operation in which the medical device 1 moves in a direction to pull out the catheter 11 from the target are performed. The movement of the moving stage 2a is controlled by the control device 3.

The medical device 1 includes a wire driving unit or portion (a linear member driving portion, a line driving portion, or a main body driving portion) 300 for driving the catheter 11. In at least one embodiment, the medical device 1 is a robot catheter device which drives the catheter 11 with use of the wire driving unit or portion 300 controlled by the control device 3.

The control device 3 is able to control the wire driving unit or portion 300 to perform an operation for bending the catheter 11. In at least one embodiment, the wire driving unit or portion 300 is built in the base unit or portion 200. More specifically, the base unit or portion 200 includes a base housing 200f, which stores the wire driving unit or portion 300 therein. Thus, the base unit or portion 200 includes the wire driving unit or portion 300. The wire driving unit or portion 300 and the base unit or portion 200 may be collectively referred to as a “catheter driving device (a base device or a main body)” for one or more embodiments.

With regard to the extension direction of the catheter 11, an end portion at which a fore-end or front end of the catheter 11 to be inserted into a target is arranged is referred to as a “distal end”. With regard to the extension direction of the catheter 11, an end opposite to the distal end is referred to as a “proximal end”.

The catheter unit or portion 100 includes a proximal end cover 16, which covers the proximal end side of the catheter 11. The proximal end cover 16 has a tool hole 16a. A medical tool is able to be inserted into the catheter 11 via the tool hole 16a.

As mentioned above, in at least one embodiment, the catheter 11 has at least a function of serving as a guide device for guiding a medical tool to a desired target position inside a target.

For example, in a state in which an endoscope has been inserted into the catheter 11, the user inserts the catheter 11 to the target position inside a target. At this time, at least one of a manual operation of the user, a movement of the moving stage 2a, and the driving of the catheter 11 by the wire driving unit or portion 300 is used. After the catheter 11 arrives at the target position, the endoscope is pulled out from the catheter 11 via the tool hole 16a. Then, the user inserts the medical tool via the tool hole 16a, so that an operation, such as, but not limited to, extraction of various samples from the inside of the target or treatment for the inside of the target, is performed.

As described below, the catheter unit or portion 100 is detachably mounted to the catheter driving device (the base device or the main body), more specifically, to the base unit or portion 200 in one or more embodiments. After the medical device 1 is used, the user is able to detach the catheter unit or portion 100 from the base unit or portion 200, attach a new catheter unit or portion 100 to the base unit or portion 200, and use the medical device 1 again.

As illustrated in FIG. 2, the medical device 1 includes an operation unit or portion 400. In at least one embodiment, the operation unit or portion 400 is provided at the catheter unit or portion 100. The operation unit or portion 400 is operated by the user when or in a case where the catheter unit or portion 100 is being fixed to the base unit or portion 200 or in a case where detachment of the catheter unit or portion 100 from the base unit or portion 200 is performed.

### Catheter Embodiment Examples

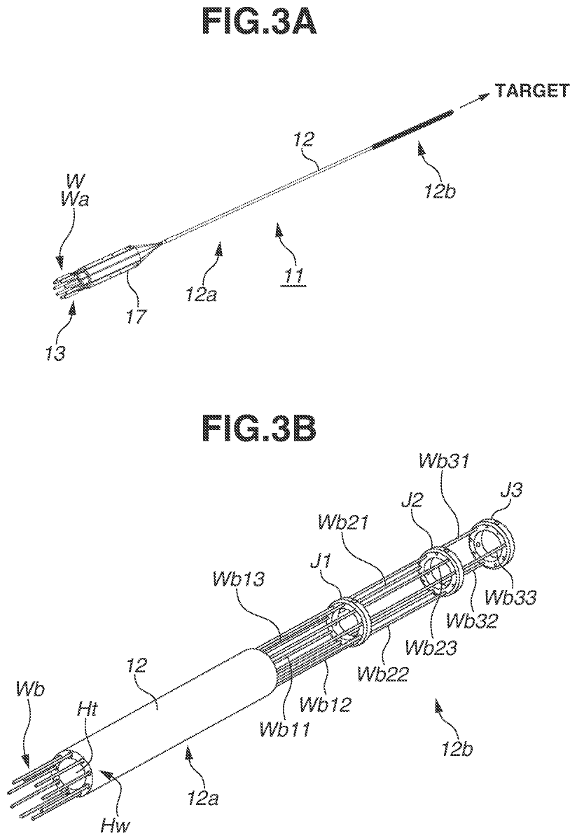

The catheter 11, which serves as a bendable body, is described with reference to FIGS. 3A and 3B. FIGS. 3A and 3B are explanatory diagrams of the catheter 11. FIG. 3A is a diagram used to explain at least one embodiment of the entire catheter 11. FIG. 3B is an enlarged view of the catheter 11.

The catheter 11 includes a bending portion (a bending body or a catheter main body) 12 and a bending driving portion (a catheter driving portion) 13, which is configured to bend the bending portion 12. The bending driving portion 13 is configured to receive a driving force from the wire driving unit or portion 300 via a coupling device 21 described below to bend the bending portion 12.

The catheter 11 is extended along an insertion direction of the catheter 11 into a target.

The extension direction (longitudinal direction) of the catheter 11 is the same as the extension direction (longitudinal direction) of the bending portion 12 and the extension direction (longitudinal direction) of each of first to ninth driving wires (W11 to W33 as shown in the embodiment of FIG. 3A) described below.

The bending driving portion 13 includes a plurality of driving wires (driving lines, linear members, or linear actuators) connected to the bending portion 12. Specifically, the bending driving portion 13 includes a first driving wire W11, a second driving wire W12, a third driving wire W13, a fourth driving wire W21, a fifth driving wire W22, a sixth driving wire W23, a seventh driving wire W31, an eighth driving wire W32, and a ninth driving wire W33 (see e.g., FIG. 3A).

Each of the first to ninth driving wires (W11 to W33) includes a held portion (a held shaft or a held rod) Wa (see e.g., FIG. 3A). Specifically, the first driving wire W11 includes a first held portion Wa11. The second driving wire W12 includes a second held portion Wa12. The third driving wire W13 includes a third held portion Wa13. The fourth driving wire W21 includes a fourth held portion Wa21. The fifth driving wire W22 includes a fifth held portion Wa22. The sixth driving wire W23 includes a sixth held portion Wa23. The seventh driving wire W31 includes a seventh held portion Wa31. The eighth driving wire W32 includes an eighth held portion Wa32. The ninth driving wire W33 includes a ninth held portion Wa33.

In at least one embodiment, the first to ninth held portions (Wa11 to Wa33) have the same shape.

Each of the first to ninth driving wires (W11 to W33) includes a wire body (a line body or a linear body) Wb having flexibility (see e.g., FIG. 3B). Specifically, the first driving wire W11 includes a first wire body Wb11. The second driving wire W12 includes a second wire body Wb12. The third driving wire W13 includes a third wire body Wb13. The fourth driving wire W21 includes a fourth wire body Wb21.

The fifth driving wire W22 includes a fifth wire body Wb22. The sixth driving wire W23 includes a sixth wire body Wb23. The seventh driving wire W31 includes a seventh wire body Wb31. The eighth driving wire W32 includes an eighth wire body Wb32. The ninth driving wire W33 includes a ninth wire body Wb33.

In at least one embodiment, the first to third wire bodies (Wb11 to Wb13) have the same shape. The fourth to sixth wire bodies (Wb21 to Wb23) have the same shape.

The seventh to ninth wire bodies (Wb31 to Wb33) have the same shape in one or more embodiments. In one or more embodiments, the first to ninth wire bodies (Wb11 to Wb33) have the same shape excluding their lengths.

The first to ninth held portions (Wa11 to Wa33) are fixed to the first to ninth wire bodies (Wb11 to Wb33) at the proximal ends of the first to ninth wire bodies (Wb11 to Wb33), respectively.

The first to ninth driving wires (W11 to W33) are inserted into the bending portion 12 via a wire guide 17, and the first to ninth driving wires (W11 to W33) are fixed to the bending portion 12.

In at least one embodiment, the material of each of the first to ninth driving wires (W11 to W33) is metal. However, the material of each of the first to ninth driving wires (W11 to W33) may be resin. The material of each of the first to ninth driving wires (W11 to W33) may contain or include metal or resin.

An optional one of the first to ninth driving wires (W11 to W33) may be referred to as a “driving wire W” such that the “driving wire W” may refer to one or more of the first to ninth driving wires (W11 to W33). In at least one embodiment, the first to ninth driving wires (W11 to W33) have the same shape excluding the respective lengths of the first to ninth wire bodies (Wb11 to Wb33), which may vary in length.

In at least one embodiment, the bending portion 12 is a tubular member having flexibility and having a path Ht into which to insert a medical tool (see e.g., FIG. 3B).

The wall surface of the bending portion 12 is provided with a plurality of wire holes for the passage of the respective first to ninth driving wires (W11 to W33). Specifically, the wall surface of the bending portion 12 is provided with a first wire hole Hw11, a second wire hole Hw12, a third wire hole Hw13, a fourth wire hole Hw21, a fifth wire hole Hw22, a sixth wire hole Hw23, a seventh wire hole Hw31, an eighth wire hole Hw32, and a ninth wire hole Hw33. The first to ninth wire holes Hw (Hw11 to Hw33) correspond to the first to ninth driving wires (W11 to W33), respectively. A number following the reference character “Hw” represents a number of the corresponding driving wire. For example, the first driving wire W11 is inserted into the first wire hole Hw11.

An optional one of the first to ninth wire holes (Hw11 to Hw33) may be referred to as a “wire hole Hw” such that “wire hole Hw” may refer to one or more of the first to ninth wire holes (Hw11 to Hw33). In at least one embodiment, the first to ninth wire holes (Hw11 to Hw33) have the same shape.

The bending portion 12 includes an intermediate region 12a and a winding region 12b. The winding region 12b is arranged at the distal end of the bending portion 12, and a first guide ring J1, a second guide ring J2, and a third guide ring J3 are arranged at the winding region 12b. The winding region 12b means a region capable of controlling the magnitude or direction of bending of the bending portion 12 by the bending driving portion 13 moving the first guide ring J1, the second guide ring J2, and the third guide ring J3. In FIG. 3B, a part of the bending portion 12 covering the first to third guide rings (J1 to J3) is omitted from illustration to see the internal structure of the bending portion 12. As shown, for example, in FIG. 3A, the bending portion 12 preferably has a covering and/or a wall surface for the first to third guide rings (J1 to J3).

In at least one embodiment, the bending portion 12 includes a plurality of auxiliary rings (not illustrated). In the winding region 12b, the first guide ring J1, the second guide ring J2, and the third guide ring J3 are fixed to the wall surface of the bending portion 12. In at least one embodiment, the plurality of auxiliary rings is arranged at the proximal end side of the first guide ring J1, between the first guide ring J1 and the second guide ring J2, and between the second guide ring J2 and the third guide ring J3.

The medical tool may be guided to the fore-end or front end of the catheter 11 by the path Ht, the first to third guide rings (J1 to J3), and the plurality of auxiliary rings.

The first to ninth driving wires (W11 to W33) are fixed to the first to third guide rings (J1 to J3) via, or while passing through, the intermediate region 12a.

Specifically, in at least one embodiment, the first driving wire W11, the second driving wire W12, and the third driving wire W13 are fixed to the first guide ring J1 while being passed through the plurality of auxiliary rings. The fourth driving wire W21, the fifth driving wire W22, and the sixth driving wire W23 are fixed to the second guide ring J2 while being passed through the first guide ring J1 and the plurality of auxiliary rings. The seventh driving wire W31, the eighth driving wire W32, and the ninth driving wire W33 are fixed to the third guide ring J3 while being passed through the first guide ring J1, the second guide ring J2, and the plurality of auxiliary rings.

The medical device 1 is able to cause the wire driving unit or portion 300 to drive the bending driving portion 13, thus bending the bending portion 12 toward a direction intersecting with the extension direction of the catheter 11. Specifically, the medical device 1 is able to move each of, or one or more of, the first to ninth driving wires (W11 to W33) along the extension direction of the bending portion 12, thus bending the winding region 12b of the bending portion 12 in a direction intersecting with the extension direction of the catheter 11.

The user is able to use at least one of movement of the medical device 1 by a manual operation or the moving stage 2a and bending of the bending portion 12, thus inserting the catheter 11 into an intended portion inside a target.

Furthermore, while, in at least one embodiment, the first to ninth driving wires (W11 to W33) are used to move the first to third guide rings (J1 to J3) to bend the bending portion 12, the at least one embodiment is not limited to this configuration. Any one or any two of the first to third guide rings (J1 to J3) and the driving wires fixed thereto may be omitted from the configuration.

For example, a configuration in which, in the catheter 11, the first to sixth driving wires (W11 to W23) and the first and second guide rings (J1 and J2) are omitted and only the seventh to ninth driving wires (W31 to W33) and the third guide ring J3 are included may be employed in one or more embodiments. Moreover, a configuration in which, in the catheter 11, the first to third driving wires (W11 to W13) and the first guide ring J1 are omitted and only the fourth to ninth driving wires (W21 to W33) and the second and third guide rings (J2 and J3) are included may also be employed.

Moreover, a configuration in which, in the catheter 11, one guide ring is driven by two driving wires may be employed. In this case, the number of guide rings may also be one or may also be larger than one.

### Catheter Unit or Portion Embodiment Examples

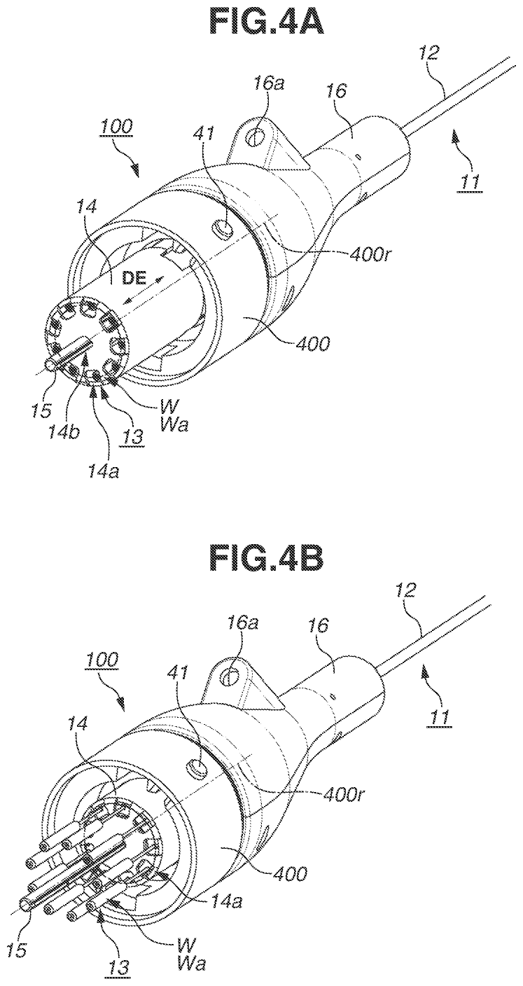

At least one embodiment of the catheter unit or portion 100 is described with reference to FIGS. 4A and 4B.

FIGS. 4A and 4B are explanatory diagrams of the catheter unit or portion 100. FIG. 4A is an explanatory diagram of the catheter unit or portion 100 in a state in which a wire cover 14 described below is in a covering position. FIG. 4B is an explanatory diagram of the catheter unit or portion 100 in a state in which the wire cover 14 described below is in a retracted position.

The catheter unit or portion 100 includes the catheter 11, which includes the bending portion 12 and the bending driving portion 13, and the proximal end cover 16, which supports the proximal end of the catheter 11. The catheter unit or portion 100 further includes a cover (a wire cover) 14 for covering and protecting the first to ninth driving wires (W11 to W33) serving as a plurality of driving wires.

The catheter unit or portion 100 is attachable to and detachable from the base unit or portion 200 along attachment and detachment directions DE. The mounting direction of the catheter unit or portion 100 to the base unit or portion 200 and the demounting direction of the catheter unit or portion 100 from the base unit or portion 200 are parallel to the attachment and detachment directions DE.

The proximal end cover (a frame body, a bending portion housing, or a catheter housing) 16 is a cover which covers a part of the catheter 11. The proximal end cover 16 has a tool hole 16a via which to insert a medical tool into the path Ht of the bending portion 12.

The wire cover 14 is provided with a plurality of wire cover holes (cover holes) 14a (see e.g., FIGS. 4A-4B) for the passage of the respective first to ninth driving wires (W11 to W33). Specifically, the wire cover 14 is provided with a first wire cover hole 14a11, a second wire cover hole 14a12, a third wire cover hole 14a13, a fourth wire cover hole 14a21, a fifth wire cover hole 14a22, a sixth wire cover hole 14a23, a seventh wire cover hole 14a31, an eighth wire cover hole 14a32, and a ninth wire cover hole 14a33. The first to ninth wire cover holes (14a11 to 14a33) correspond to the first to ninth driving wires (W11 to W33), respectively. A number following the reference character “14a” represents a number of the corresponding driving wire. For example, the first driving wire W11 is inserted into the first wire cover hole 14a11.

An optional one of the first to ninth wire cover holes (14a11 to 14a33) may be referred to as a “wire cover hole 14a” such that “wire cover hole 14a” may refer to one or more of the first to ninth wire cover holes (14a11 to 14a33). In at least one embodiment, the first to ninth wire cover holes (14a11 to 14a33) have the same shape.

The wire cover 14 is able to move between a covering position (see FIG. 4A) in which the wire cover 14 covers the first to ninth driving wires (W11 to W33) and a retracted position (see FIG. 4B) in which the wire cover 14 is retracted from the covering position. The retracted position can also be referred to as an “exposure position” in which the wire cover 14 causes the first to ninth driving wires (W11 to W33) to be exposed.

Before the catheter unit or portion 100 is attached to the base unit or portion 200, the wire cover 14 is situated in the covering position. In a case where the catheter unit or portion 100 is attached to the base unit or portion 200, the wire cover 14 moves from the covering position to the retracted position along the attachment and detachment directions DE.

In at least one embodiment, after moving from the covering position to the retracted position, the wire cover 14 may be kept in the retracted position. Accordingly, even in a case where, after the catheter unit or portion 100 is attached to the base unit or portion 200, the catheter unit or portion 100 is detached from the base unit or portion 200, the wire cover 14 may be kept in the retracted position.

However, the wire cover 14 may be configured to return to the covering position after moving from the covering position to the retracted position. For example, the catheter unit or portion 100 may include an urging member which urges the wire cover 14 from the retracted position toward the covering position. In this case, if, after the catheter unit or portion 100 is attached to the base unit or portion 200, the catheter unit or portion 100 is detached from the base unit or portion 200, the wire cover 14 may be moved from the retracted position to the covering position.

When or in a case where the wire cover 14 is in the retracted position, the first to ninth held portions (Wa11 to Wa33) of the first to ninth driving wires (W11 to W33) project with respect to the wire cover 14. As a result, coupling between the bending driving portion 13 and the coupling device 21 described below is allowed. When or in a case where the wire cover 14 is in the retracted position, the first to ninth held portions (Wa11 to Wa33) of the first to ninth driving wires (W11 to W33) project from the first to ninth wire cover holes (14a11 to 14a33). More specifically, the first to ninth held portions (Wa11 to Wa33) project from the first to ninth wire cover holes (14a11 to 14a33) in an attachment direction Da described below.

As illustrated in FIG. 4B, the first to ninth driving wires (W11 to W33) are arranged side by side along a circle (a virtual circle) having a predetermined radius. In the present disclosure, this circle is referred to as a “pitch circle” for one or more embodiments.

In at least one embodiment, the catheter unit or portion 100 includes a key shaft (a key or a catheter-side key) 15. In at least one embodiment, the key shaft 15 extends along the attachment and detachment directions DE. The wire cover 14 is provided with a shaft hole 14b through which the key shaft 15 passes (see e.g., FIG. 4A).

The key shaft 15 is able to engage with a key receiving portion 22 described below. The key shaft 15 engaging with the key receiving portion 22 causes the movement of the catheter unit or portion 100 with respect to the base unit or portion 200 to be limited in a predetermined range with respect to the circumferential direction of a circle (virtual circle) along which the first to ninth driving wires (W11 to W33) are arranged side by side.

In at least one embodiment, as viewed along the attachment and detachment directions DE, the first to ninth driving wires (W11 to W33) are arranged outside the key shaft 15 in such a way as to surround the key shaft 15. In other words, the key shaft 15 is arranged radially inside a circle (virtual circle) along which the first to ninth driving wires (W11 to W33) are arranged side by side.

In at least one embodiment, the catheter unit or portion 100 includes an operation unit or portion 400. The operation unit or portion 400 is configured to be movable (rotatable) with respect to the proximal end cover 16 and the bending driving portion 13.

The operation unit or portion 400 is rotatable around a rotational axis 400r. The rotational axis 400r of the operation unit or portion 400 extends along the attachment and detachment directions DE.

The operation unit or portion 400 is configured to be movable (rotatable) with respect to the base unit or portion 200 in a state in which the catheter unit or portion 100 is attached to the base unit or portion 200. More specifically, the operation unit or portion 400 is configured to be movable (rotatable) with respect to the base housing 200f, the wire driving unit or portion 300, and the coupling device 21 described below.

### Base Unit or Portion Embodiment Examples

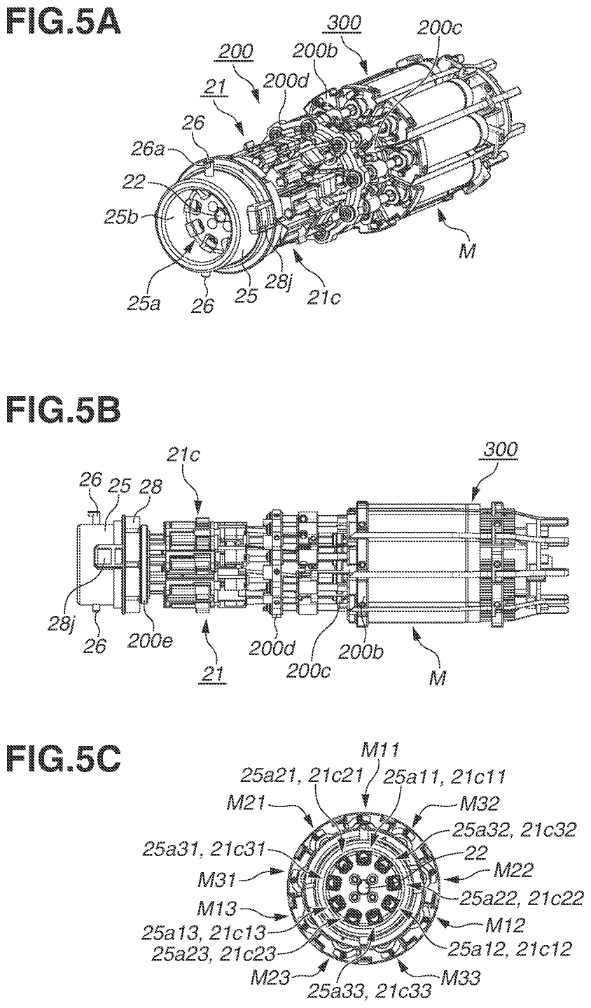

The base unit or portion 200 and the wire driving unit or portion 300 are described with reference to FIGS. 5A, 5B, and 5C.

FIGS. 5A to 5C are explanatory diagrams of the base unit or portion 200 and the wire driving unit or portion 300. FIG. 5A is a perspective view illustrating an internal structure of the base unit or portion 200. FIG. 5B is a side view illustrating the internal structure of the base unit or portion 200. FIG. 5C is a diagram illustrating the base unit or portion 200 as viewed along the attachment and detachment directions DE.

As mentioned above, the medical device 1 includes the base unit or portion 200 and the wire driving unit or portion 300. In at least one embodiment, the wire driving unit or portion 300 is housed in the base housing 200f and is thus arranged inside the base unit or portion 200. In other words, the base unit or portion 200 includes the wire driving unit or portion 300 in one or more embodiments.

The wire driving unit or portion 300 includes a plurality of drive sources (motors). In at least one embodiment, the wire driving unit or portion 300 includes a first drive source M11, a second drive source M12, a third drive source M13, a fourth drive source M21, a fifth drive source M22, a sixth drive source M23, a seventh drive source M31, an eighth drive source M32, and a ninth drive source M33 (see e.g., FIGS. 5A-5C).

An optional one of the first to ninth drive sources (M11 to M33) may be referred to as a “drive source M” such that “drive source M” may refer to one or more of the first to ninth drive sources (M11 to M33). In at least one embodiment, the first to ninth drive sources (M11 to M33) have the same configuration.

The base unit or portion 200 includes the coupling device 21. The coupling device 21 is housed in the base housing 200f. The coupling device 21 is connected to the wire driving unit or portion 300. The coupling device 21 includes a plurality of coupling portions. In the present exemplary embodiment, the coupling device 21 includes a first coupling portion 21c11, a second coupling portion 21c12, a third coupling portion 21c13, a fourth coupling portion 21c21, a fifth coupling portion 21c22, a sixth coupling portion 21c23, a seventh coupling portion 21c31, an eighth coupling portion 21c32, and a ninth coupling portion 21c33.

An optional one of the first to ninth coupling portions (21c11 to 21c33) can be referred to as a “coupling portion 21c” such that “coupling portion 21c” may refer to one or more of the first to ninth coupling portions (21c11 to 21c33). In at least one embodiment, the first to ninth coupling portions (21c11 to 21c33) have the same configuration.

The plurality of coupling portions 21c is connected to the plurality of drive sources M, respectively, and the plurality of coupling portions 21c is driven by the plurality of drive sources M, respectively. Specifically, in one or more embodiments, the first coupling portion 21c11 is connected to the first drive source M11 and is driven by the first drive source M11. The second coupling portion 21c12 is connected to the second drive source M12 and is driven by the second drive source M12. The third coupling portion 21c13 is connected to the third drive source M13 and is driven by the third drive source M13. The fourth coupling portion 21c21 is connected to the fourth drive source M21 and is driven by the fourth drive source M21. The fifth coupling portion 21c22 is connected to the fifth drive source M22 and is driven by the fifth drive source M22. The sixth coupling portion 21c23 is connected to the sixth drive source M23 and is driven by the sixth drive source M23. The seventh coupling portion 21c31 is connected to the seventh drive source M31 and is driven by the seventh drive source M31. The eighth coupling portion 21c32 is connected to the eighth drive source M32 and is driven by the eighth drive source M32. The ninth coupling portion 21c33 is connected to the ninth drive source M33 and is driven by the ninth drive source M33.

As described below, the bending driving portion 13, which includes the first to ninth driving wires (W11 to W33), is coupled to the coupling device 21. The bending driving portion 13 receives a driving force from the wire driving unit or portion 300 via the coupling device 21, which may be used to bend the bending portion 12.

The driving wire W is coupled to the coupling portion 21c via the held portion Wa. The plurality of driving wires W is coupled to the plurality of coupling portions 21c, respectively.

Specifically, the first held portion Wa11 of the first driving wire W11 is coupled to the first coupling portion 21c11. The second held portion Wa12 of the second driving wire W12 is coupled to the second coupling portion 21c12. The third held portion Wa13 of the third driving wire W13 is coupled to the third coupling portion 21c13. The fourth held portion Wa21 of the fourth driving wire W21 is coupled to the fourth coupling portion 21c21. The fifth held portion Wa22 of the fifth driving wire W22 is coupled to the fifth coupling portion 21c22. The sixth held portion Wa23 of the sixth driving wire W23 is coupled to the sixth coupling portion 21c23. The seventh held portion Wa31 of the seventh driving wire W31 is coupled to the seventh coupling portion 21c31. The eighth held portion Wa32 of the eighth driving wire W32 is coupled to the eighth coupling portion 21c32. The ninth held portion Wa33 of the ninth driving wire W33 is coupled to the ninth coupling portion 21c33.

The base unit or portion 200 includes a base frame 25. The base frame 25 is provided with a plurality of insertion holes 25a for the passage of the respective first to ninth driving wires (W11 to W33). Specifically, the base frame 25 is provided with a first insertion hole 25a11, a second insertion hole 25a12, a third insertion hole 25a13, a fourth insertion hole 25a21, a fifth insertion hole 25a22, a sixth insertion hole 25a23, a seventh insertion hole 25a31, an eighth insertion hole 25a32, and a ninth insertion hole 25a33. The first to ninth insertion holes (25a11 to 25a33) correspond to the first to ninth driving wires (W11 to W33), respectively. A number following reference character “25a” represents a number of the corresponding driving wire. For example, the first driving wire W11 is inserted into the first insertion hole 25a11.

An optional one of the first to ninth insertion holes (25a11 to 25a33) may be referred to as an “insertion hole 25a” such that “insertion hole 25a” may refer to one or more of the first to ninth insertion holes (25a11 to 25a33). In at least one embodiment, the first to ninth insertion holes (25a11 to 25a33) have the same shape.

The base frame 25 is provided with an attachment opening 25b, into which the wire cover 14 is inserted. The first to ninth insertion holes (25a11 to 25a33) are arranged at the bottom portion of the attachment opening 25b.

Additionally, the base unit or portion 200 includes a motor frame 200b, a first bearing frame 200c, a second bearing frame 200d, and a third bearing frame 200e in at least one embodiment (see e.g., FIG. 5B).

The base frame 25 includes a key receiving portion (a key hole, a base-side key, or a main body-side key) 22 which receives the key shaft 15. In response to the key shaft 15 and the key receiving portion 22 engaging with each other, the catheter unit or portion 100 is attached to the base unit or portion 200 in a correct phase.

In response to the key shaft 15 and the key receiving portion 22 engaging with each other, the movement of the catheter unit or portion 100 relative to the base unit or portion 200 is limited in a predetermined range with regard to the circumferential direction of a circle (virtual circle) along which the first to ninth driving wires (W11 to W33) are arranged side by side.

As a result, the first to ninth driving wires (W11 to W33) engage with the corresponding first to ninth insertion holes (25a11 to 25a33) and the corresponding first to ninth coupling portions (21c11 to 21c33), respectively. In other words, the driving wire W is prevented from engaging with a not corresponding insertion hole 25 and a not corresponding coupling portion 21.

The user is able to cause the key shaft 15 and the key receiving portion 22 to engage with each other, thus correctly coupling the first to ninth driving wires (W11 to W33) with the first to ninth coupling portions (21c11 to 21c33), respectively. Accordingly, the user is able to easily mount the catheter unit or portion 100 to the base unit or portion 200.

In at least one embodiment, the key shaft 15 has a raised portion projecting in a direction intersecting with the attachment and detachment directions DE, and the key receiving portion 22 has a recessed portion into which the raised portion is inserted. The position at which the raised portion and the recessed portion engage with each other in the circumferential direction is the position at which the driving wire W engages with the corresponding insertion hole 25a and the corresponding coupling portion 21c.

Furthermore, the key shaft 15 may be arranged at any one of the base unit or portion 200 and the catheter unit or portion 100, and the key receiving portion 22 may be arranged at the other thereof. For example, the key shaft 15 may be arranged at the side of the base unit or portion 200 and the key receiving portion 22 may be arranged at the side of the catheter unit or portion 100.

### Coupling Between Motor and Driving Wire in One or More Embodiments

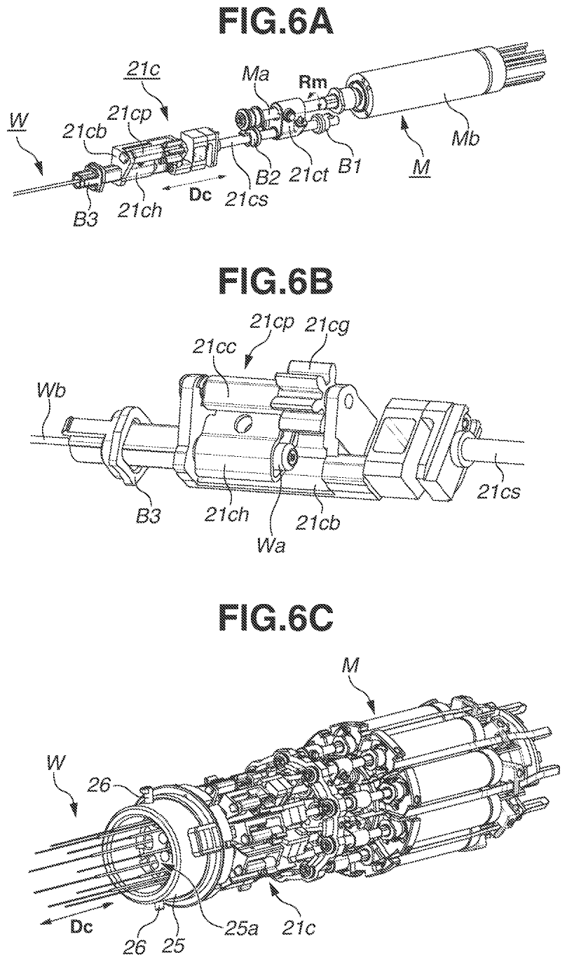

Coupling between the wire driving unit or portion 300, the coupling device 21, and the bending driving portion 13 is described with reference to FIGS. 6A, 6B, and 6C.

FIGS. 6A to 6C are explanatory diagrams of the wire driving unit or portion 300, the coupling device 21, and the bending driving portion 13. FIG. 6A is a perspective view of the drive source M, the coupling portion 21c, and the driving wire W. FIG. 6B is an enlarged view of the coupling portion 21c and the driving wire W. FIG. 6C is a perspective view illustrating coupling between the wire driving unit or portion 300, the coupling device 21, and the bending driving portion 13.

In at least one embodiment, respective configurations in which the first to ninth driving wires (W11 to W33) and the first to ninth coupling portions (21c11 to 21c33) are coupled to each other are the same. Moreover, respective configurations in which the first to ninth coupling portions (21c11 to 21c33) and the first to ninth drive sources (M11 to M33) are connected to each other are the same. Accordingly, in the following description, one driving wire W, one coupling portion 21c, and one drive source M are used to describe a configuration in which the one driving wire W, the one coupling portion 21c, and the one drive source M are connected to each other.

As illustrated in FIG. 6A, the drive source M includes an output shaft Ma, which is a motor shaft, and a motor main body Mb, which rotates the output shaft Ma in rotational directions Rm. The surface of the output shaft Ma is provided with a spiral groove. The output shaft Ma has what is called a screw shape. The motor main body Mb is fixed to the motor frame 200b.

The coupling portion 21c includes a tractor 21ct, which is connected to the output shaft Ma, and a tractor supporting shaft 21cs, which supports the tractor 21ct. The tractor supporting shaft 21cs is connected to a coupling base 21cb.

The coupling portion 21c includes a plate spring 21ch, which serves as a holding portion for holding the held portion Wa of the driving wire W. The driving wire W is in engagement with the coupling portion 21c via the insertion hole 25a.

More specifically, the held portion Wa engages with the plate spring 21ch. As described below, the plate spring 21ch is able to assume a state in which the plate spring 21ch pinches and fixes the held portion Wa (a fixing state) and a state in which the plate spring 21ch has released the held portion Wa (a releasing state).

The coupling portion 21c includes a pressing member 21cp. As illustrated in FIG. 6B, the pressing member 21cp includes a gear portion 21cg, which meshes with an internal gear 29 described below, and a cam 21cc, which serves as a pressing portion for pressing the plate spring 21ch.

As described below, the cam 21cc is able to move relative to the plate spring 21ch. In response to the cam 21cc moving, the plate spring 21ch is switched between the fixing state and the releasing state.

The coupling portion 21c is supported by a first bearing B1, a second bearing B2, and a third bearing B3. The first bearing B1 is supported by the first bearing frame 200c of the base unit or portion 200. The second bearing B2 is supported by the second bearing frame 200d of the base unit or portion 200. The third bearing B3 is supported by the third bearing frame 200e of the base unit or portion 200. Accordingly, the coupling portion 21c is restricted from rotating around the output shaft Ma when or in a case where the output shaft Ma has rotated in the rotational directions Rm. Furthermore, the first bearing B1, the second bearing B2, and the third bearing B3 are provided with respect to each of the first to ninth coupling portions (21c11 to 21c33).

Since the coupling portion 21c is restricted from rotating around the output shaft Ma, when or in a case where the output shaft Ma rotates, the spiral groove of the output shaft Ma causes a force along the rotational axis direction of the output shaft Ma to act on the tractor 21ct. As a result, the coupling portion 21c moves (in directions Dc) along the rotational axis direction of the output shaft Ma. In response to the coupling portion 21c moving, the driving wire W moves, thus bending the bending portion 12.

Thus, the output shaft Ma and the tractor 21ct constitute what is called a feed screw which converts rotational motion transmitted from the drive source M into rectilinear motion by screw threads. In at least one embodiment, the output shaft Ma and the tractor 21ct use a slide screw, but may use a ball screw in one or more other embodiments.

As illustrated in FIG. 6C, in response to the catheter unit or portion 100 being attached to the base unit or portion 200, the first to ninth driving wires (W11 to W33) are coupled to the first to ninth coupling portions (21c11 to 21c33), respectively.

The control device 3 is able to control each of the first to ninth drive sources (M11 to M33) independently.

Thus, an optional drive source of the first to ninth drive sources (M11 to M33) is able to operate or stop independently regardless of whether the other drive sources are in a stopped state. In other words, the control device 3 is able to control each of the first to ninth driving wires (W11 to W33) independently. As a result, each of the first to third guide rings (J1 to J3) is controlled independently, so that the winding region 12b of the bending portion 12 is able to bend in an optional direction.

### Mounting of Catheter Unit or Portion in One or More Embodiments

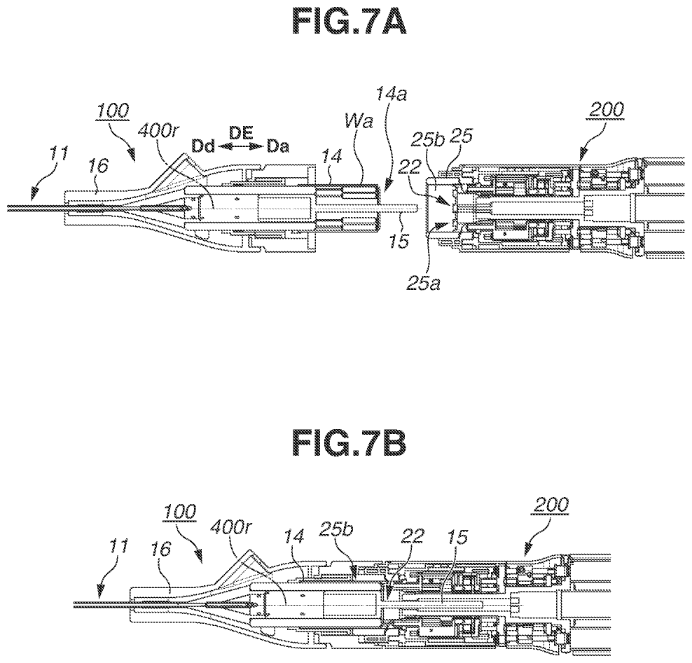

An operation for mounting the catheter unit or portion 100 to the base unit or portion 200 is described with reference to FIGS. 7A and 7B.

FIGS. 7A and 7B are explanatory diagrams used to explain mounting of the catheter unit or portion 100. FIG. 7A is a diagram illustrating a state obtained before the catheter unit or portion 100 is mounted to the base unit or portion 200. FIG. 7B is a diagram illustrating a state obtained after the catheter unit or portion 100 is mounted to the base unit or portion 200.

In at least one embodiment, the attachment and detachment directions DE of the catheter unit or portion 100 are identical with the direction of the rotational axis 400r of the operation unit or portion 400. Out of the attachment and detachment directions DE, a direction in which to attach the catheter unit or portion 100 to the base unit or portion 200 is referred to as an “attachment direction Da”. Out of the attachment and detachment directions DE, a direction in which to detach the catheter unit or portion 100 from the base unit or portion 200 (a direction opposite to the attachment direction Da) is referred to as a “detachment direction Dd”.

As illustrated in FIG. 7A, in a state obtained before the catheter unit or portion 100 is mounted to the base unit or portion 200, the wire cover 14 is situated at the covering position. At this time, the wire cover 14 covers the first to ninth driving wires (W11 to W33) in such a way as to prevent the first to ninth held portions (Wa11 to Wa33) from projecting from the first to ninth wire cover holes (14a11 to 14a33) of the wire cover 14.

When or in a case where the key shaft 15 and the key receiving portion 22 engage with each other and the catheter unit or portion 100 is moved in the attachment direction Da relative to the base unit or portion 200, the catheter unit or portion 100 is attached to the base unit or portion 200. The catheter unit or portion 100 being attached to the base unit or portion 200 causes the wire cover 14 to move to the retracted position. In at least one embodiment, the wire cover 14 comes into abutting contact with the base frame 25 and thus moves from the covering position to the retracted position (see FIG. 7B).

More specifically, when or in a case where the catheter unit or portion 100 is attached, the wire cover 14 comes into abutting contact with the base frame 25 and thus stops. In this state, in response to the catheter unit or portion 100 being moved in the attachment direction Da, in the catheter unit or portion 100, the wire cover 14 relatively moves with respect to portions other than the wire cover 14. As a result, the wire cover 14 moves from the covering position to the retracted position.

While the wire cover 14 moves from the covering position to the retracted position, the held portion Wa of the driving wire W projects from the wire cover hole 14a of the wire cover 14 and is then inserted into the insertion hole 25a. Then, the held portion Wa engages with the plate spring 21ch of the coupling portion 21c (see FIG. 6B).

In a state in which the catheter unit or portion 100 is only attached to the base unit or portion 200, it is possible to detach the catheter unit or portion 100 by moving the catheter unit or portion 100 in the detachment direction Dd relative to the base unit or portion 200. Moreover, as described below, a state in which the catheter unit or portion 100 is only attached to the base unit or portion 200 brings about a state in which fixing between the driving wire W and the coupling portion 21c has been released.

Operating the operation unit or portion 400 in a state in which the catheter unit or portion 100 is attached to the base unit or portion 200 prevents the catheter unit or portion 100 from being detached from the base unit or portion 200. Additionally, operating the operation unit or portion 400 in a state in which the catheter unit or portion 100 is attached to the base unit or portion 200 causes the bending driving portion 13 to be fixed to the coupling device 21, so that the bending driving portion 13 is coupled to the wire driving unit or portion 300 via the coupling device 21.

### Fixing of Bending Driving Portion and Releasing of Fixing Thereof in One or More Embodiments

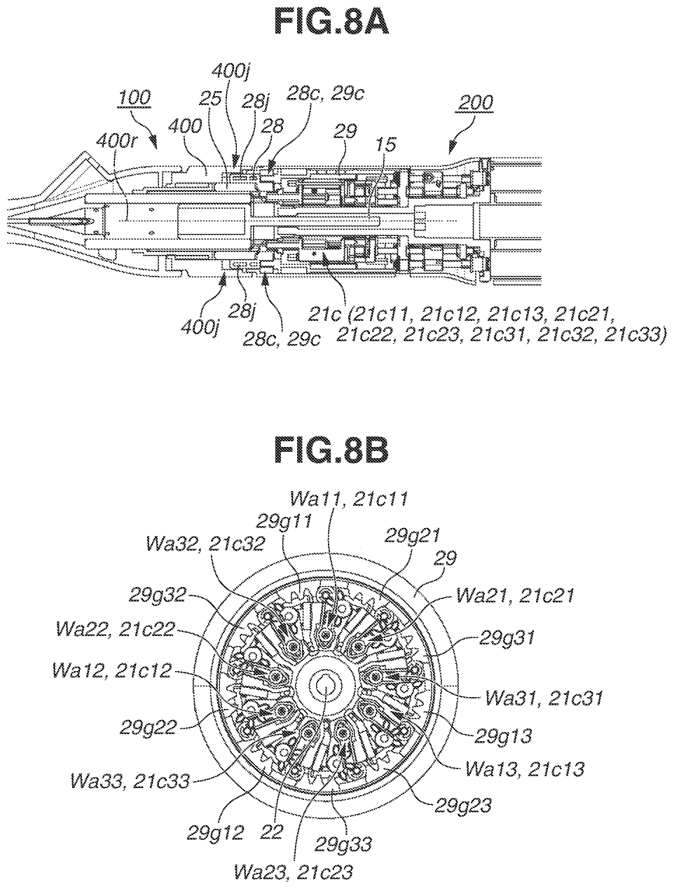

A configuration for fixing the bending driving portion 13 to the coupling device 21 and a configuration for releasing fixing of the bending driving portion 13 by the coupling device 21 are described with reference to FIGS. 8A and 8B, FIG. 9, FIG. 10, FIG. 11, FIG. 12, FIG. 13, and FIG. 14.

FIGS. 8A and 8B are diagrams used to explain fixing between the catheter unit or portion 100 and the base unit or portion 200. FIG. 8A is a sectional view of the catheter unit or portion 100 and the base unit or portion 200. Thus, FIG. 8A is a sectional view obtained by cutting the catheter unit or portion 100 and the base unit or portion 200 along the rotational axis 400r. FIG. 8B is a sectional view of the base unit or portion 200. Thus, FIG. 8B is a sectional view obtained by cutting the base unit or portion 200 at the coupling portion 21c in a direction perpendicular to the rotational axis 400r.

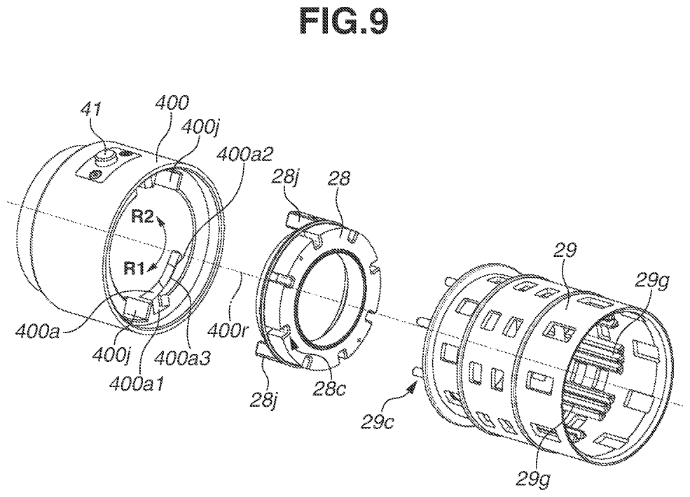

FIG. 9 is an exploded view used to explain coupling between the catheter unit or portion 100 and the base unit or portion 200.

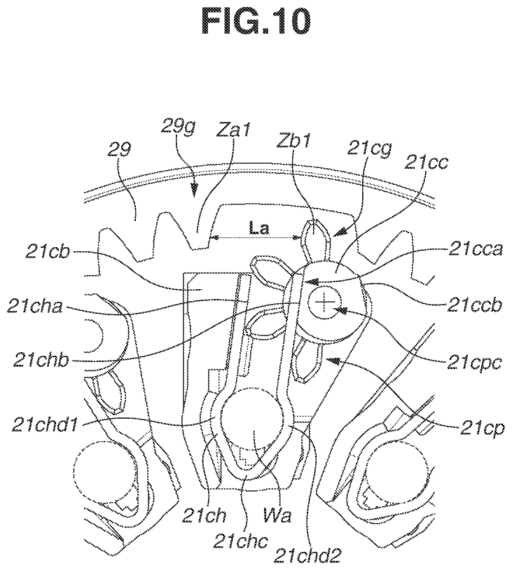

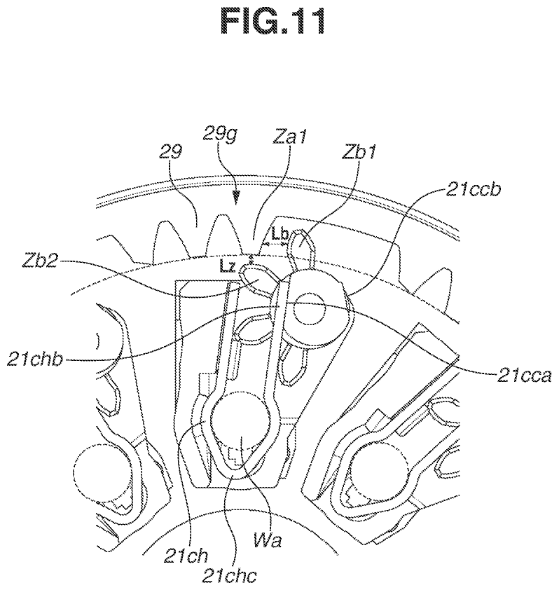

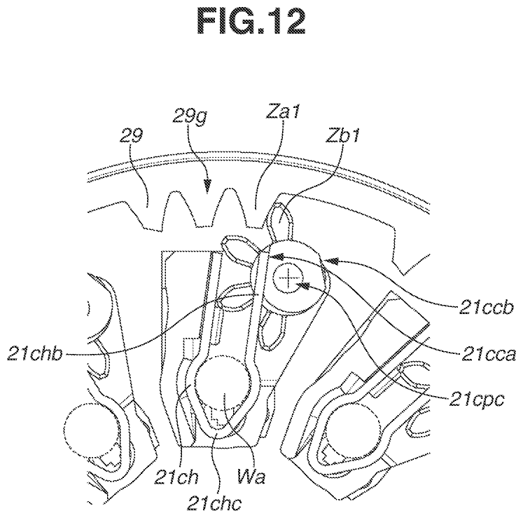

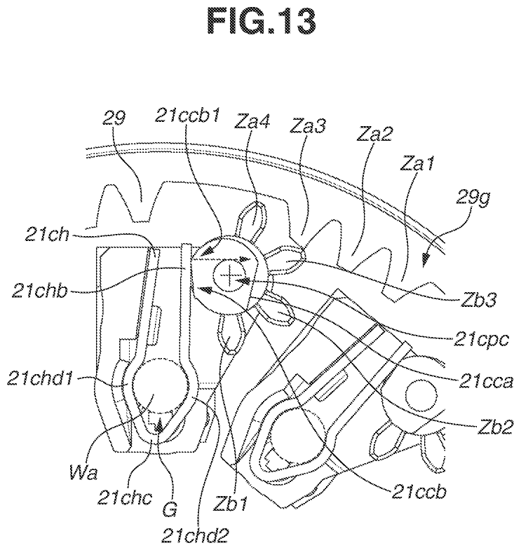

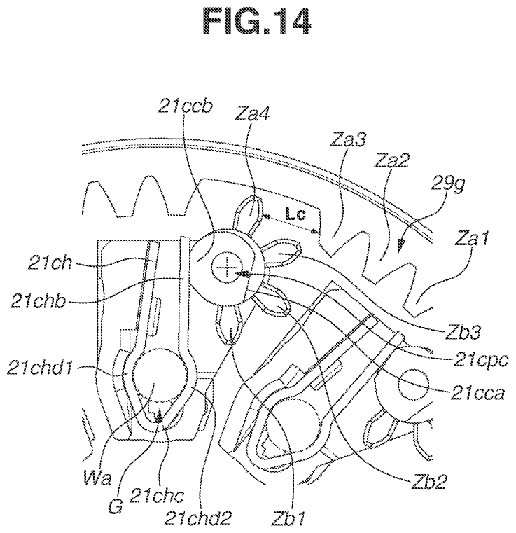

FIG. 10, FIG. 11, FIG. 12, FIG. 13, and FIG. 14 are diagrams used to explain fixing of the driving wire W by the coupling portion 21c.

As illustrated in FIG. 8A and FIG. 9, the base unit or portion 200 includes a joint (an intermediate member or a second transmission member) 28 and an internal gear 29 serving as a moving gear (an interlocking gear, a transmission member, or a first transmission member) which interlocks with the operation unit or portion 400 via the joint 28.

The joint 28 includes a plurality of transmission portions 28c, and the internal gear 29 includes a plurality of transmission receiving portions 29c. The plurality of transmission portions 28c is in engagement with the plurality of transmission receiving portions 29c, and, when or in a case where the joint 28 rotates, the rotation of the joint 28 is transmitted to the internal gear 29.

In response to the catheter unit or portion 100 being attached to the base unit or portion 200, an engagement portion 400j included in the operation unit or portion 400 engages with a joint engagement portion 28j of the joint 28. When or in a case where the operation unit or portion 400 rotates, the rotation of the operation unit or portion 400 is transmitted to the joint 28. The operation unit or portion 400, the joint 28, and the internal gear 29 rotate in the same direction.

The internal gear 29 includes a plurality of tooth portions for switching between a state in which the first to ninth coupling portions (21c11 to 21c33) fix the first to ninth driving wires (W11 to W33), respectively, and a state in which the first to ninth coupling portions (21c11 to 21c33) release the first to ninth driving wires (W11 to W33), respectively. The plurality of tooth portions of the internal gear 29 engages with gear portions 21cg of pressing members 21cp included in the first to ninth coupling portions (21c11 to 21c33), respectively.

Specifically, in at least one embodiment, the internal gear 29 includes a first tooth portion 29g11, a second tooth portion 29g12, a third tooth portion 29g13, a fourth tooth portion 29g21, a fifth tooth portion 29g22, a sixth tooth portion 29g23, a seventh tooth portion 29g31, an eighth tooth portion 29g32, and a ninth tooth portion 29g33. The first to ninth tooth portions (29g11 to 29g33) are formed with respective gaps arranged therebetween.

The first tooth portion 29g11 meshes with the gear portion 21cg of the first coupling portion 21c11. The second tooth portion 29g12 meshes with the gear portion 21cg of the second coupling portion 21c12. The third tooth portion 29g13 meshes with the gear portion 21cg of the third coupling portion 21c13. The fourth tooth portion 29g21 meshes with the gear portion 21cg of the fourth coupling portion 21c21. The fifth tooth portion 29g22 meshes with the gear portion 21cg of the fifth coupling portion 21c22. The sixth tooth portion 29g23 meshes with the gear portion 21cg of the sixth coupling portion 21c23. The seventh tooth portion 29g31 meshes with the gear portion 21cg of the seventh coupling portion 21c31. The eighth tooth portion 29g32 meshes with the gear portion 21cg of the eighth coupling portion 21c32. The ninth tooth portion 29g33 meshes with the gear portion 21cg of the ninth coupling portion 21c33.

An optional one of the first to ninth tooth portions (29g11 to 29g33) may be referred to as a “tooth portion 29g” such that “tooth portion 29g” may refer to one or more of the first to ninth tooth portions 29g (29g11 to 29g33) for the first to ninth coupling portions (21c11 to 21c33). In at least one embodiment, the first to ninth tooth portions (29g11 to 29g33) have the same configuration.

In at least one embodiment, respective configurations in which the first to ninth driving wires (W11 to W33) are coupled to the first to ninth coupling portions (21c11 to 21c33), respectively, are the same. Moreover, respective configurations in which the first to ninth coupling portions (21c11 to 21c33) are connected to the first to ninth tooth portions (29g11 to 29g33), respectively, are the same. Accordingly, in the following description, a configuration in which one driving wire W, one coupling portion 21c, and one tooth portion 29g are connected to each other is described with use of the one driving wire W, the one coupling portion 21c, and the one tooth portion 29g.

In each of the first to ninth coupling portions (21c11 to 21c33), in response to the gear portion 21cg being moved by the internal gear 29, the pressing member 21cp rotates, so that the cam 21cc moves between the pressing position and the retracted position, which is retracted from the pressing position.

In response to the operation unit or portion 400 being rotated, the internal gear 29 rotates. In response to the internal gear 29 rotating, each of the first to ninth coupling portions (21c11 to 21c33) operates.

In a state in which the catheter unit or portion 100 is mounted to the base unit or portion 200, the operation unit or portion 400 is able to move between a fixing position (locking position) and a detachment position. Moreover, as described below, in a state in which the catheter unit or portion 100 is mounted to the base unit or portion 200, the operation unit or portion 400 is able to move to a releasing position. With regard to the rotational direction of the operation unit or portion 400, the releasing position is located between the fixing position and the detachment position. In a state in which the operation unit or portion 400 is situated in the detachment position, the catheter unit or portion 100 is attached to the base unit or portion 200.

In a state in which the catheter unit or portion 100 is attached to the base unit or portion 200, the driving wire W is in the state of not being fixed (locked) to the coupling portion 21c. This state is referred to as a “releasing state of the coupling portion 21c”. Furthermore, a state in which the driving wire W is fixed (locked) to the coupling portion 21c is referred to as a “locking state of the coupling portion 21c”.

An operation for fixing the driving wire W to the coupling portion 21c is described with reference to FIG. 10, FIG. 11, FIG. 12, FIG. 13, and FIG. 14.

In a state obtained before the operation unit or portion 400 is operated after the catheter unit or portion 100 is attached to the base unit or portion 200, the catheter unit or portion 100 is able to be detached from the base unit or portion 200. In the following description, a state in which the catheter unit or portion 100 is able to be detached from the base unit or portion 200 is referred to as a “detachable state”.

FIG. 10 is a diagram illustrating states of the internal gear 29 and the coupling portion 21c when or in a case where the catheter unit or portion 100 is in the detachable state. Thus, FIG. 10 is a diagram illustrating the internal gear 29 and the coupling portion 21c in a state in which the operation unit or portion 400 is situated in the detachment position.

The plate spring 21ch of the coupling portion 21c includes a fixed portion 21cha, which is fixed to the coupling base 21cb, and a pressed portion 21chb, which is in abutting contact with the cam 21cc of the pressing member 21cp. The plate spring 21ch includes a first portion 21chd1 and a second portion 21chd2. In response to the catheter unit or portion 100 being attached to the base unit or portion 200, the held portion Wa is inserted into between the first portion 21chd1 and the second portion 21chd2.

The cam 21cc includes a holding surface 21cca and a pressing surface 21ccb. With regard to the rotational radial direction of the pressing member 21cp, the holding surface 21cca is situated at a position closer to the rotational center 21cpc of the pressing member 21cp than the pressing surface 21ccb.

As illustrated in FIG. 10, when or in a case where the operation unit or portion 400 is in the detachable state (a state in which the operation unit or portion 400 is in the detachment position), the plate spring 21ch is held at a position where the pressed portion 21chb is in abutting contact with the holding surface 21cca. Moreover, the tooth Za1 of the internal gear 29 and the tooth Zb1 of the gear portion 21cg are stopped in a state in which a clearance La has occurred therebetween.

With regard to rotational directions of the operation unit or portion 400, a direction in which the operation unit or portion 400 moves from the detachment position to the releasing position and the fixing position is referred to as a “locking direction (fixing direction)”, and a direction in which the operation unit or portion 400 moves from the fixing position to the releasing position and the detachment position is referred to as a “releasing direction”. The operation unit or portion 400 rotates in the releasing direction from the releasing position and thus moves to the detachment position. The operation unit or portion 400 rotates in the locking direction from the releasing position and thus moves to the fixing position.

In a state in which the catheter unit or portion 100 is attached to the base unit or portion 200 and the operation unit or portion 400 is in the detachment position, the coupling portion 21c is in the releasing state, so that fixing of the driving wire W by the coupling portion 21c is released.

When or in a case where the coupling portion 21c is in the releasing state, the cam 21cc is situated in the retracted position, in which the cam 21cc has been retracted from a pressing position described below. At this time, a state in which fixing of the held portion Wa by the plate spring 21ch has been released occurs. A force with which the first portion 21chd1 and the second portion 21chd2 constrict the held portion Wa when or in a case where the coupling portion 21c is in the releasing state is smaller than a force with which the first portion 21chd1 and the second portion 21chd2 constrict the held portion Wa when or in a case where the coupling portion 21c is in the locking state.

In a case where, when or in a case where the coupling portion 21c is in the releasing state, the catheter unit or portion 100 is moved in the detachment direction Dd relative to the base unit or portion 200, it is possible to extract the held portion Wa from between the first portion 21chd1 and the second portion 21chd2.

When or in a case where the coupling portion 21c is in the releasing state, a state in which a force with which the first portion 21chd1 and the second portion 21chd2 constrict the held portion Wa does not occur (a state in which the magnitude of the force is zero) is favorable. When or in a case where the coupling portion 21c is in the releasing state, it is favorable that a gap occurs between at least one of the first portion 21chd1 and the second portion 21chd2 and the held portion Wa.

FIG. 11 is a diagram illustrating states of the internal gear 29 and the coupling portion 21c when or in a case where the operation unit or portion 400 has rotated in the locking direction from the detachment position. Thus, FIG. 11 is a diagram illustrating states of the internal gear 29 and the coupling portion 21c in a state in which the operation unit or portion 400 is in the releasing position.

When or in a case where, in a state in which the operation unit or portion 400 is in the detachment position (FIG. 10), the operation unit or portion 400 is rotated in the locking direction, the internal gear 29 rotates clockwise. Then, the operation unit or portion 400 is situated in the releasing position.

Furthermore, since, even in a case where the operation unit or portion 400 is rotated, the key shaft 15 and the key receiving portion 22 are in engagement with each other, the entirety of the catheter unit or portion 100 (excluding the operation unit or portion 400) is restricted from rotating relative to the base unit or portion 200. Thus, in a state in which the entirety of the catheter unit or portion 100 (excluding the operation unit or portion 400) and the base unit or portion 200 have been stopped, the operation unit or portion 400 is able to rotate relative to them.

In response to the internal gear 29 rotating clockwise, the clearance between the tooth Za1 of the internal gear 29 and the tooth Zb1 of the gear portion 21cg decreases from the clearance La to a clearance Lb.

A tooth Zb2 of the gear portion 21cg is arranged at a position having a clearance Lz from an addendum circle (indicated by dashed line) of the tooth portion 29g of the internal gear 29. Therefore, the internal gear 29 is able to rotate without interfering with the tooth Zb2. On the other hand, the coupling portion 21c is kept in the same state (releasing state) as the state illustrated in FIG. 10.

When or in a case where the operation unit or portion 400 is further rotated in the locking direction from the state illustrated in FIG. 11, the internal gear 29 further rotates clockwise. The states of the internal gear 29 and the coupling portion 21c obtained at that time is illustrated in FIG. 12.

FIG. 12 is a diagram illustrating states of the internal gear 29 and the coupling portion 21c when or in a case where the operation unit or portion 400 has been rotated in the locking direction from the releasing position.

As illustrated in FIG. 12, in response to the operation unit or portion 400 rotating in the locking direction from the releasing position, the tooth Za1 of the internal gear 29 and the tooth Zb1 of the gear portion 21cg come into contact with each other. On the other hand, the coupling portion 21c is kept in the same state as the state illustrated in FIG. 10 and FIG. 11, i.e., the releasing state.

FIG. 13 is a diagram illustrating a state in which, in response to the operation unit or portion 400 rotating in the locking direction, the pressing member 21cp has rotated.

As illustrated in FIG. 13, when or in a case where the operation unit or portion 400 is further rotated in the locking direction from the state illustrated in FIG. 12, the internal gear 29 further rotates clockwise.

In response to the internal gear 29 moving from the state illustrated in FIG. 12 to the state illustrated in FIG. 13, the internal gear 29 causes the gear portion 21cg to rotate clockwise. In response to the gear portion 21cg rotating, the holding surface 21cca moves away from the pressed portion 21chb and the pressing surface 21ccb comes close to the pressed portion 21chb. Then, pinching of the held portion Wa by the first portion 21chd1 and the second portion 21chd2 is started.

Then, while the pressed portion 21chb is being pressed by a corner portion 21ccb1 arranged at the end portion of the pressing surface 21ccb, the tooth Za3 of the internal gear 29 moves to a position where the tooth Za3 moves away from the tooth Zb3 of the gear portion 21cg. At this time, a state in which the held portion Wa is pinched by the first portion 21chd1 and the second portion 21chd2 occurs.

When or in a case where the tooth Za3 of the internal gear 29 has moved away from the tooth Zb3 of the gear portion 21cg, transmission of a driving force from the internal gear 29 to the gear portion 21cg ends. At this time, the cam 21cc is in a state in which the corner portion 21ccb1 receives a reaction force from the plate spring 21ch.

With regard to the rotational radial direction of the pressing member 21cp, a reaction force of the plate spring 21ch acting on the corner portion 21ccb1 acts on a position away from the rotational center 21cpc of the pressing member 21cp, so that the pressing member 21cp rotates clockwise. At this time, the pressing member 21cp rotates in the same direction as the direction in which the pressing member 21cp is caused to rotate by the internal gear 29 rotating clockwise.

FIG. 14 is a diagram illustrating states of the internal gear 29 and the coupling portion 21c in a state in which the operation unit or portion 400 is in the fixing position.

As illustrated in FIG. 14, upon receiving a reaction force of the plate spring 21ch, the pressing member 21cp further rotates from the state illustrated in FIG. 13.

As illustrated in FIG. 14, the pressing member 21cp stops in a state in which the pressing surface 21ccb of the cam 21cc and the pressed portion 21chb of the plate spring 21ch have come into surface contact with each other. Thus, a state in which the pressing surface 21ccb and the surface of the pressed portion 21chb are situated side by side on the same plane.

At this time, the coupling portion 21c is in the locking state. When or in a case where the coupling portion 21c is in the locking state, the cam 21cc of the pressing member 21cp is situated in the pressing position, so that the pressing surface 21ccb presses the pressed portion 21chb.

When or in a case where the coupling portion 21c is in the locking state, the held portion Wa is pinched by the first portion 21chd1 and the second portion 21chd2. Thus, the plate spring 21ch is pressed by the cam 21cc, and the held portion Wa is constricted by the plate spring 21ch. As a result, the held portion Wa is fixed by the plate spring 21ch.

In at least one embodiment, in the plate spring 21ch, the first portion 21chd1 and the second portion 21chd2 press the held portion Wa at respective positions away from each other. Additionally, a bent portion 21chc interconnecting the first portion 21chd1 and the second portion 21chd2 is arranged between the first portion 21chd1 and the second portion 21chd2. The bent portion 21chc is arranged with a gap G from the held portion Wa. This enables the held portion Wa to be stably fixed by the first portion 21chd1 and the second portion 21chd2.

While a resin or metal may be used as the material of the plate spring 21ch, it is favorable that a metal is used in one or more embodiments.

When or in a case where the coupling portion 21c is in the locking state, extracting the held portion Wa from between the first portion 21chd1 and the second portion 21chd2 is restricted.

Furthermore, the tooth Za3 of the internal gear 29 and the tooth Zb4 of the gear portion 21cg are stopped at respective positions where a clearance Lc occurs therebetween.

To release fixing between the driving wire W and the coupling portion 21c, the user rotates the operation unit or portion 400, which has been in the fixing position, in the releasing direction. At this time, the internal gear 29 rotates counterclockwise from the state illustrated in FIG. 14. In response to the internal gear 29 rotating counterclockwise, the tooth Za3 of the internal gear 29 comes into abutting contact with the tooth Zb4 of the gear portion 21cg, so that the pressing member 21cp is rotated counterclockwise.

In response to the internal gear 29 further rotating counterclockwise, fixing of the driving wire W by the coupling portion 21c is released. Operations of the internal gear 29 and the pressing member 21cp at this time are operations opposite to the above-mentioned operations thereof. Thus, fixing of the driving wire W by the coupling portion 21c is released by the operations opposite to the above-mentioned operations for fixing the driving wire W by the coupling portion 21c.

The above-mentioned operations are performed in each of the first to ninth coupling portions (21c11 to 21c33).

Thus, in the process in which the operation unit or portion 400 moves from the detachment position to the fixing position, in response to the operation unit or portion 400 moving (rotating), the first to ninth coupling portions (21c11 to 21c33) transition from the releasing state to the locking state. In the process in which the operation unit or portion 400 moves from the fixing position to the detachment position, in response to the operation unit or portion 400 moving (e.g., rotating), the first to ninth coupling portions (21c11 to 21c33) transition from the locking state to the releasing state.

A state in which the first to ninth driving wires (W11 to W33) have been fixed by the first to ninth coupling portions (21c11 to 21c33), respectively, is referred to as a “first state”. A state in which fixing of the first to ninth driving wires (W11 to W33) by the first to ninth coupling portions (21c11 to 21c33), respectively, has been released is referred to as a “second state”.

The first state and the second state are switched in conjunction with the movement of the operation unit or portion 400. Thus, the first state and the second state are switched in conjunction with the operation unit or portion 400 moving between the detachment position and the fixing position.

The internal gear 29 is configured to interlock with the operation unit or portion 400. In at least one embodiment, the joint 28 functions as a transmission member for causing the operation unit or portion 400 and the internal gear 29 to interlock with each other. The internal gear 29 and the joint 28 have a function serving as an interlocking portion which interlocks with the operation unit or portion 400 in such a manner that the first state and the second state are switched in conjunction with the movement of the operation unit or portion 400.

Specifically, in a state in which the catheter unit or portion 100 is attached to the base unit or portion 200, the internal gear 29 and the joint 28 cause a part of the plate spring 21ch (the pressed portion 21chb) to move relative to the held portion Wa in conjunction with the movement of the operation unit or portion 400. In response to the pressed portion 21chb moving, the locking state and the releasing state of the coupling portion 21c are switched.

Furthermore, a configuration in which the internal gear 29 is directly moved by the operation unit or portion 400 may be employed. In that case, the internal gear 29 has a function serving as an interlocking portion.

### Movement of Operation Unit or Portion in One or More Embodiments

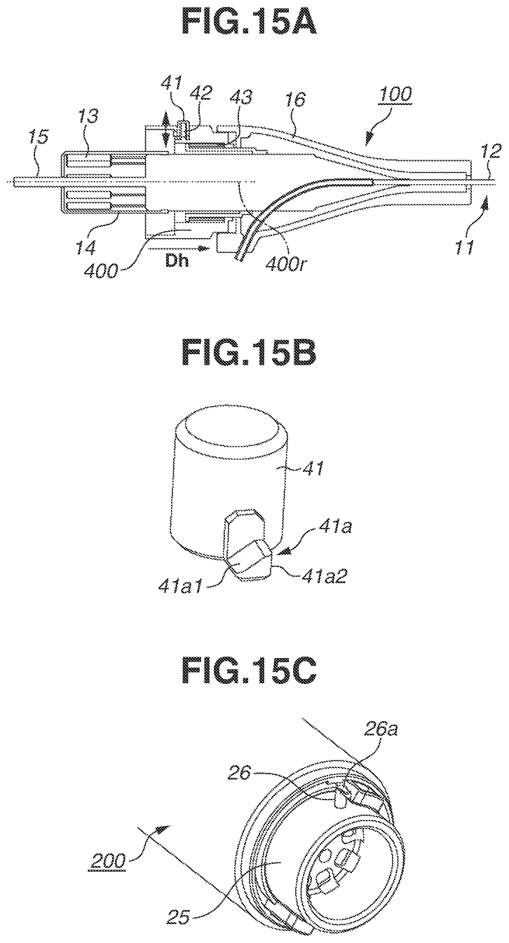

The movement of the operation unit or portion 400 is described with reference to FIGS. 15A, 15B, and 15C, FIGS. 16A, 16B, and 16C, and FIGS. 17A, 17B, and 17C.

In at least one embodiment, in a state in which the catheter unit or portion 100 is attached to the base unit or portion 200, the operation unit or portion 400 is configured to be able to move between the detachment position, the releasing position, and the fixing position. The releasing position is situated between the detachment position and the fixing position.

In at least one embodiment, the first state and the second state are switched in conjunction with the movement of the operation unit or portion 400 between the releasing position and the fixing position of the operation unit or portion 400.

In at least one embodiment, the operation unit or portion 400 is able to move between the detachment position and fixing position by moving in directions different from the attachment and detachment directions DE. The operation unit or portion 400 moves between the detachment position and the fixing position by moving in directions intersecting with the attachment and detachment directions DE (favorably, in directions perpendicular to the attachment and detachment directions DE). In at least one embodiment, the operation unit or portion 400 moves between the detachment position and the fixing position by rotating around the rotational axis 400R extending in the attachment and detachment directions DE.

FIGS. 15A to 15C are explanatory diagrams of the catheter unit or portion 100 and the base unit or portion 200. FIG. 15A is a sectional view of the catheter unit or portion 100. FIG. 15B is a perspective view of a button 41. FIG. 15C is a perspective view of the base unit or portion 200.

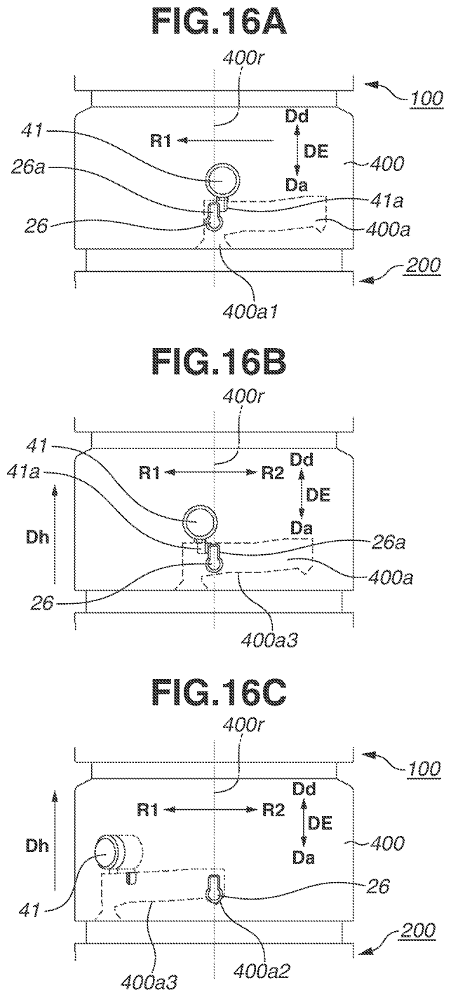

FIGS. 16A to 16C are diagrams used to explain operations of the operation unit or portion 400. FIG. 16A is a diagram illustrating a state in which the operation unit or portion 400 is in the detachment position. FIG. 16B is a diagram illustrating a state in which the operation unit or portion 400 is in the releasing position. FIG. 16C is a diagram illustrating a state in which the operation unit or portion 400 is in the fixing position.

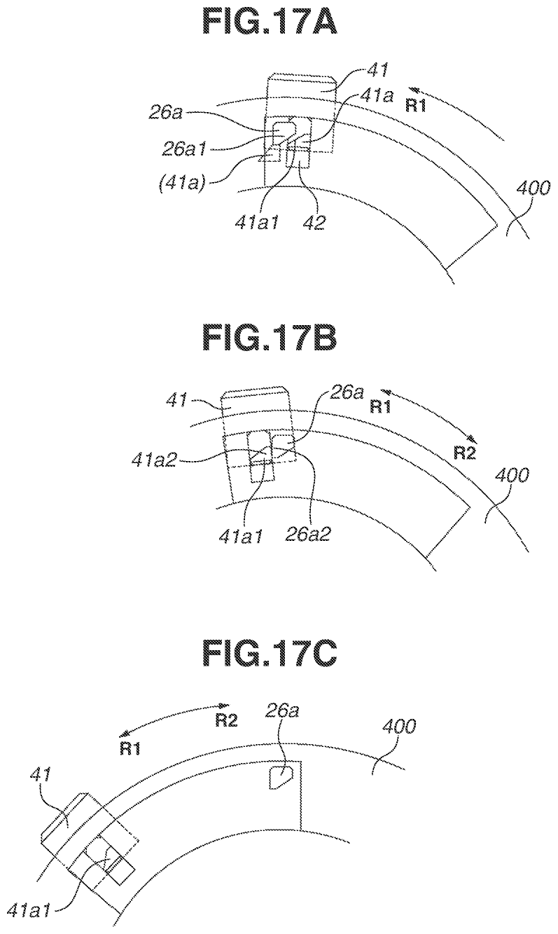

FIGS. 17A to 17C are sectional views used to explain operations of the operation unit or portion 400. FIG. 17A is a sectional view illustrating a state in which the operation unit or portion 400 is in the detachment position. FIG. 17B is a sectional view illustrating a state in which the operation unit or portion 400 is in the releasing position. FIG. 17C is a sectional view illustrating a state in which the operation unit or portion 400 is in the fixing position.

When or in a case where the operation unit or portion 400 is in the fixing position, the coupling portion 21c is in the locking state and the held portion Wa of the driving wire W is fixed to the corresponding coupling portion 21c (see FIG. 14).

When or in a case where the operation unit or portion 400 is in the releasing position, the coupling portion 21c is in the releasing state and locking between the held portion Wa of the driving wire W and the coupling portion 21c is released (see FIG. 11). In this state, connection between the driving wire W and the wire driving unit or portion 300 is severed. Accordingly, when or in a case where the catheter 11 has received external force, the bending portion 12 is able to be freely bent without the resistance of the wire driving unit or portion 300.

When or in a case where the operation unit or portion 400 is in the detachment position, the catheter unit or portion 100 is allowed to be detached from the base unit or portion 200. Moreover, in a state in which the operation unit or portion 400 is in the detachment position, the catheter unit or portion 100 is able to be attached to the base unit or portion 200. When or in a case where the operation unit or portion 400 is in the detachment position, the coupling portion 21c is in the releasing state and locking between the held portion Wa of the driving wire W and the coupling portion 21c is released (see FIG. 10).

As illustrated in FIG. 15A, the catheter unit or portion 100 includes an operation unit or portion urging spring 43, which urges the operation unit or portion 400, a button 41, which is a moving member, and a button spring 42, which urges the button 41.

In at least one embodiment, the operation unit or portion urging spring 43 is a compressed spring. The operation unit or portion 400 is urged by the operation unit or portion urging spring 43 in a direction Dh to come close to the proximal end cover 16.

In at least one embodiment, the button 41 and the button spring 42 are provided at the operation unit or portion 400. When or in a case where the operation unit or portion 400 moves to the detachment position, the releasing position, or the fixing position, the button 41 and the button spring 42 move together with the operation unit or portion 400.

The button 41 is configured to be able to move relative to the operation unit or portion 400 in directions intersecting with the direction of the rotational axis 400r of the operation unit or portion 400. The button 41 is urged by the button spring 42 toward outside the catheter unit or portion 100 (in a direction to move away from the rotational axis 400r).

As described below, the operation unit or portion 400 is restricted by the button 41 from moving from the releasing position to the detachment position. Moreover, in response to the button 41 being moved relative to the operation unit or portion 400, the operation unit or portion 400 is allowed to move from the releasing position to the detachment position.

The button 41 includes a button projection (a restricted portion) 41a. The button projection 41a includes a button slant surface 41a1 and a restricted surface 41a2.

The base unit or portion 200 includes a base frame 25. A locking shaft 26 is provided at the base frame 25. The locking shaft 26 includes a locking projection (a restricting portion) 26a.

In at least one embodiment, the locking shaft 26 includes a plurality of locking shafts (in at least one embodiment, two locking shafts). All of the locking shafts 26 can include respective locking projections 26a, and some locking shafts 26 can include respective locking projections 26a.

On the other hand, as illustrated in FIG. 9, FIG. 16A, FIG. 16B, and FIG. 16C, a locking groove 400a, which engages with the locking shaft 26, is provided at the inner side of the operation unit or portion 400. The locking groove 400a extends in a direction different from the attachment and detachment directions DE. In at least one embodiment, the locking groove 400a extends in a rotational direction of the operation unit or portion 400. It can be said that the locking groove 400a extends in a direction intersecting with the attachment and detachment directions DE (in a direction perpendicular to the attachment and detachment directions DE).

In a case where a plurality of locking shafts 26 is provided, the locking groove 400a is provided for each of the plurality of locking shafts 26.

As illustrated in FIG. 16A, when or in a case where the catheter unit or portion 100 is attached to the base unit or portion 200, the locking shaft 26 engages with the locking groove 400a via an entrance 400a1 of the locking groove 400a.

At this time, the operation unit or portion 400 is situated in the detachment position and the coupling portion 21c is in the releasing state (see FIG. 10). Accordingly, a state in which fixing of the first to ninth driving wires (W11 to W33) by the first to ninth coupling portions (21c11 to 21c33), respectively, is released occurs. Moreover, as illustrated in FIG. 17A, the button projection 41a and the locking projection 26a face each other.

When or in a case where the operation unit or portion 400 is rotated in a locking direction R1 in a state in which the operation unit or portion 400 is in the detachment position, the button slant surface 41a1 of the button projection 41a comes into abutting contact with a slant surface 26a1 of the locking projection 26a. Against the urging force of the button spring 42, the button 41 moves toward the inner side of the operation unit or portion 400 (in a direction to come close to the rotational axis 400r). Then, the button projection 41a climbs over the locking projection 26a, and the operation unit or portion 400 moves to the releasing position (see FIG. 17B).

At this time, the coupling portion 21c is in the releasing state (see FIG. 11). Accordingly, a state in which fixing of the first to ninth driving wires (W11 to W33) by the first to ninth coupling portions (21c11 to 21c33), respectively, is released occurs.

When or in a case where the operation unit or portion 400 is rotated in the locking direction R1 in a state in which the operation unit or portion 400 is situated in the releasing position, the operation unit or portion 400 moves to the fixing position. As illustrated in FIG. 17C, in a state in which the operation unit or portion 400 is in the fixing position, a positioning portion 400a2 of the locking groove 400a is situated at a position corresponding to the locking shaft 26. The operation unit or portion 400 is being urged by the operation unit or portion urging spring 43 in the direction Dh to come close to the proximal end cover 16. As a result, the positioning portion 400a2 engages with the locking shaft 26.

In the process of the operation unit or portion 400 moving from the releasing position to the fixing position, as mentioned above, the held portion Wa of the driving wire W is fixed to the coupling portion 21c.

In a state in which the operation unit or portion 400 is situated in the fixing position, the coupling portion 21c is in the locking state (see FIG. 14). Accordingly, the first to ninth driving wires (W11 to W33) are fixed to the first to ninth coupling portions (21c11 to 21c33), respectively. In this state, a driving force from the wire driving unit or portion 300 becomes able to be transmitted to the bending driving portion 13. Thus, driving forces from the first to ninth drive sources (M11 to M33) become able to be transmitted to the first to ninth driving wires (W11 to W33) via the first to ninth coupling portions (21c11 to 21c33), respectively.

When or in a case where the operation unit or portion 400 is in the releasing position, with regard to the detachment direction Dd of the catheter unit or portion 100, a wall 400a3 for forming the locking groove 400a is situated on the upstream side of the locking shaft 26. When or in a case where the operation unit or portion 400 is in the fixing position, with regard to the detachment direction Dd, the positioning portion 400a2 is situated on the upstream side of the locking shaft 26. As a result, when or in a case where the operation unit or portion 400 is in the releasing position and when or in a case where the operation unit or portion 400 is in the fixing position, detaching the catheter unit or portion 100 from the base unit or portion 200 is restricted. On the other hand, when or in a case where the operation unit or portion 400 is in the detachment position, with regard to the detachment direction Dd, the entrance 400a1 of the locking groove 400a is situated on the upstream side of the locking shaft 26. As a result, the catheter unit or portion 100 is allowed to be detached from the base unit or portion 200.

When or in a case where, in a state in which the operation unit or portion 400 is in the fixing position, the operation unit or portion 400 is rotated in a releasing direction R2, the operation unit or portion 400 is situated in the releasing position. In the process of the operation unit or portion 400 moving from the fixing position to the releasing position, as mentioned above, the held portion Wa of the driving wire W is released from the coupling portion 21c.

In a state in which the operation unit or portion 400 is situated in the releasing position, the restricted surface 41a2 of the button projection 41a comes into abutting contact with a restricting surface 26a2 of the locking projection 26a (see FIG. 17B). In this state, rotating the operation unit or portion 400 in the releasing direction R2 is restricted. Moreover, detaching the catheter unit or portion 100 from the base unit or portion 200 is restricted.

In a state in which the operation unit or portion 400 is situated in the releasing position, in response to the user pushing the button 41 toward inside the operation unit or portion 400, the restricted surface 41a2 moves away from the restricting surface 26a2 and the button projection 41a climbs over the locking projection 26a. As a result, the operation unit or portion 400 is allowed to rotate in the releasing direction R2, so that the operation unit or portion 400 is able to move from the releasing position to the detachment position.

When or in a case where the operation unit or portion 400 has been situated in the detachment position, the coupling portion 21c enters into the releasing state.

Furthermore, in at least one embodiment, only one locking projection 26a and only one button 41 are provided. However, the medical device 1 may include a plurality of locking projections 26a and a plurality of buttons 41.

### Layout of Driving Force Transmission Mechanism in One or More Embodiments

In the following description, a layout of a driving force transmission mechanism which transmits driving force of the drive source M to the driving wire W is described for one or more embodiments.

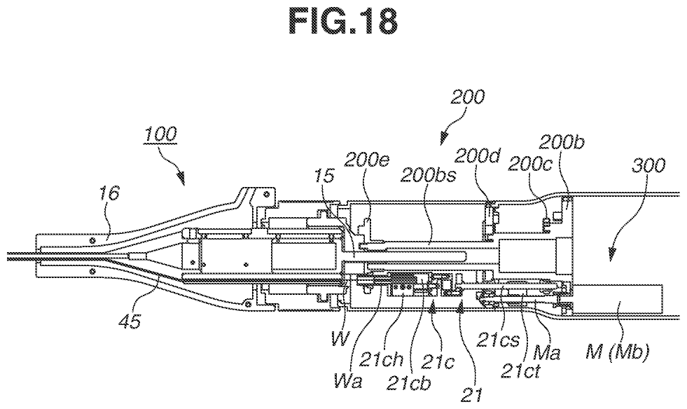

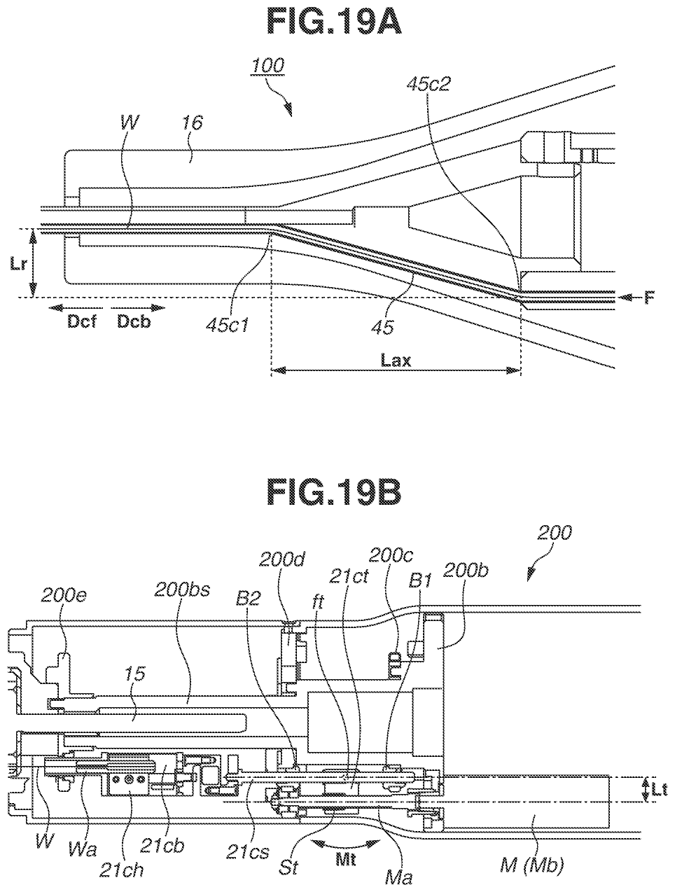

FIG. 18 is a sectional view used to explain a layout of the driving force transmission mechanism. Specifically, FIG. 18 is a sectional view taken along the rotational axis 400r in a state in which the catheter unit or portion 100 and the base unit or portion 200 are coupled to each other. In FIG. 18, parts of one of the first to ninth driving wires (W11 to W33) having the same shape, the wire driving unit or portion 300 for driving such one driving wire W, the coupling device 21, and the bending driving portion 13 are illustrated in an extracted manner. Moreover, FIGS. 19A and 19B are sectional enlarged views used to explain the layout of the driving force transmission mechanism, in which FIG. 19A is an enlarged view on the side of the catheter unit or portion 100 and FIG. 19B is an enlarged view on the side of the base unit or portion 200.

In the layout of the driving force transmission mechanism in at least one embodiment, the held portion Wa at the end portion of the driving wire W, the tractor supporting shaft 21cs, and the output shaft Ma of the drive source M are arranged as follows. The held portion Wa, the tractor supporting shaft 21cs, and the output shaft Ma each extend in an axial direction of the pitch circle, i.e., are arranged in parallel with each other. Then, the tractor supporting shaft 21cs is located at a position offset outside in the radial direction of the pitch circle with respect to the held portion Wa. Moreover, the output shaft Ma is located at a position offset outside in the radial direction of the pitch circle with respect to the tractor supporting shaft 21cs.

Thus, there are a pitch circle at which the driving wire W is provided (referred to as a “first pitch circle”) and a pitch circle which is obtained by offsetting the first pitch circle outside and at which the output shaft Ma is provided (referred to as a “second pitch circle”). Then, the tractor supporting shaft 21cs is provided along a pitch circle which is obtained by offsetting the first pitch circle outside and offsetting the second pitch circle inside (referred to as a “third pitch circle”).

In this way, a layout in which, with regard to a direction leading from the catheter 11 to the drive source M, the tractor supporting shaft 21cs is offset outside with respect to the held portion Wa and the output shaft Ma is offset outside with respect to the tractor supporting shaft 21cs is configured.

One end of the driving wire W is fixed to any one of the first to third guide rings (J1 to J3) described above with reference to FIG. 3B. In one or more embodiments, the catheter 11 is assumed to be inserted into the body of a patient, and, to enable the catheter 11 to access the finer points thereof, a plurality of driving wires W at the distal end of the catheter 11 is configured with a layout in which the diameter of the first pitch circle with the plurality of driving wires W arranged thereon becomes smaller or as small as possible.

On the other hand, at the side of the base unit or portion 200, due to, for example, the restrictions of sizes of components constituting the coupling portion 21c, which couples the driving wire W to the drive source M, the diameter of the first pitch circle is made larger. Therefore, inside the proximal end cover 16, which covers a part of the driving wire W, a bending guide 45, which guides the driving wire W in such a manner that the first pitch circle becomes larger, is arranged.

The driving wire W guided by the bending guide 45 is connected to one end of the tractor supporting shaft 21cs, which is an intermediate supporting shaft, by the held portion Wa, which is the other end not fixed by the guide rings (J1 to J3), being fixed to the coupling base 21cb and the plate spring 21ch. Furthermore, in at least one embodiment, the coupling base 21cb and the plate spring 21ch are equivalent to a first connection member which interconnects an end portion of the driving wire W and the tractor supporting shaft 21cs.

Moreover, the other end, which is not one end coupled to the driving wire W, of the tractor supporting shaft 21cs is connected to the output shaft Ma of the drive source M via the tractor 21ct. Furthermore, in at least one embodiment, the tractor 21ct is equivalent to a second connection member which interconnects the tractor supporting shaft 21cs and the output shaft Ma and converts the rotation of the output shaft Ma into rectilinear motion to cause the tractor supporting shaft 21cs to rectilinearly move.

Here, an action which a driving force generated by the drive source M applies to the driving wire W is described.

As illustrated in FIG. 19A, the bending guide 45 is a fixed pipe-like member and is configured in such a way as to enable the driving wire W to move inside the bending guide 45. Inside the bending guide 45, the driving wire W is guided in such a way as to be bent at two portions, i.e., a first bending portion 45c1 on the side of the catheter 11 and a second bending portion 45c2 on the side of the drive source M. The distance in the axial direction of the pitch circle between the first bending portion 45c1 and the second bending portion 45c2 is denoted by Lax. Upon receiving a driving force from the drive source M, the driving wire W moves in directions Dc. Furthermore, out of the directions Dc, a direction to move the driving wire W forward is referred to as a “direction Dcf” and a direction to move the driving wire W backward is referred to as a “direction Dcb”.

In this state, in the case of moving the driving wire W in the direction Dcf, with respect to an extrusion force F generated by the drive source M, a moment with the first bending portion 45c1 set as a fulcrum point and the difference Lr between radii of pitch circles obtained before and after changing formed by the driving wires W before and after bending set as the length of the moment's arm occurs. This moment causes a force for further bending the driving wire W to act.

Moreover, in the case of moving the driving wire W in the direction Dcf or the direction Dcb, due to the posture of the driving wire W being restricted, a frictional force in a direction to hinder the movement of the driving wire W occurs by the driving wire W actively coming into contact with portions around the first bending portion 45c1 and the second bending portion 45c2.

The magnitudes of these forces are in the relationship of becoming larger as the distance Lr becomes larger or the distance Lax becomes smaller, so that these forces become causes for the loss of driving force transmission to the driving wire W and for the hindrance to the smooth movement of the driving wire W.

Accordingly, in the case of guiding the driving wire W to the outer side in the radial direction of the pitch circle, it is desirable that the distance Lr be as small as possible and the distance Lax be as large as possible in one or more embodiments. However, since making the distance Lax larger may involve lengthening of the driving wire W and an increase in size of the device and may bring about another adverse effect, the distance Lr and the distance Lax may be set in such a way as to satisfy at least “Lr<Lax” in one or more embodiments.

Moreover, as illustrated in FIG. 19B, the tractor supporting shaft 21cs is in the positional relationship of being away by a distance Lt in the radial direction of the pitch circle from the output shaft Ma of the drive source M via the tractor 21ct.

As mentioned above, the tractor 21ct converts a rotational movement transmitted from the output shaft Ma of the drive source M into rectilinear motion of the tractor supporting shaft 21cs by a screw portion St. The tractor supporting shaft 21cs is constrained from moving in directions other than the directions DC in which the driving wire W moves, by the first bearing B1 and the second bearing B2. The tractor 21ct, which moves integrally with the tractor supporting shaft 21cs, receives the action of a moment Mt with a fixing portion ft for the tractor supporting shaft 21cs set as a fulcrum point, by a rectilinear force generated by the rotation of the output shaft Ma via the screw portion St. The magnitude of the moment Mt is in the relationship of being proportional to the distance Lt, so that, as the moment Mt becomes larger, a radial load which the bearings B1 and B2 supporting the tractor supporting shaft 21cs receives becomes larger. Therefore, a sliding resistance at the time of movement in the thrust direction of the tractor supporting shaft 21cs becomes large, and, as a result, the moment Mt may become a factor for hindering the smooth movement of the driving wire W.

Moreover, even in terms of abrasions of the bearings B1 and B2, an increase in load torque of the drive source M, and an abrasion by a frictional force occurring at the screw portion St, an inconvenience in which durability is inferior becomes likely to occur.

Accordingly, it is desirable that the distance Lt be set as small as possible.

Furthermore, as also illustrated in FIG. 19B, in the medical device 1 in at least one embodiment, with regard to a cross-section area perpendicular to the extension direction of the catheter 11, the size of a region in which the motor main body Mb is housed becomes largest. Generally, due to the limitations of an output torque required or that is preferably used for a motor according to specifications of the device, the minimum size of the motor may limit or affect the size of the entirety of the device. Even the present exemplary embodiment of the one or more embodiments is no exception, and the position of the output shaft Ma of the motor main body Mb is determined by the limitations in, or structural specifications of, the layout of the motor main body Mb.

In at least one embodiment, the coupling portion 21c and the drive source M are supported by a frame in such a way as to be arranged at a predetermined position on a circumference on a cross section perpendicular to the extension direction of the catheter 11. Specifically, the motor main body Mb of the drive source M is supported by the motor frame 200b, and the coupling portion 21c is supported by the first bearing frame 200c, the second bearing frame 200d, and the third bearing frame 200e.

Here, the motor frame 200b includes a cylindrical portion 200bs, and the third bearing frame 200e, which is located closest to the catheter 11 out of the frames 200b to 200e, is coupled to the outer circumference of an end portion of the cylindrical portion 200bs. The cylindrical portion 200bs is arranged in such a way as to pass through a space at the central portion of a circle occurring when or in a case where the coupling device 21 is laid out on the circumference in a cross section perpendicular to the extension direction of the catheter 11. In this way, the motor frame 200b and the third bearing frame 200e are coupled to each other via the cylindrical portion 200bs, which is a supporting portion coupling portion arranged inside the coupling portion 21c. Moreover, the first bearing frame 200c and the second bearing frame 200d are supported by the motor frame 200b. In this way, the first bearing frame 200c, the second bearing frame 200d, and the third bearing frame 200e are positioned with respect to the motor frame 200b.

Moreover, the catheter unit or portion 100 includes a key shaft 15, which is a positioning shaft. The key shaft 15 is arranged at the center of a circle in a cross section perpendicular to the extension direction of the catheter 11. In this way, the key shaft 15 is arranged inside the coupling portion 21c, inserted into the cylindrical portion 200bs of the motor frame 200b, and coupled to the cylindrical portion 200bs.

In the above-mentioned way, arranging the cylindrical portion 200bs of the motor frame 200b inside the coupling portion 21c enables securing the freedom of arrangement of a driving force transmission mechanism while preventing or reducing an increase in size of the entire device. Furthermore, in at least one embodiment, the motor frame 200b is equivalent to a first supporting member, and the third bearing frame 200e is equivalent to a second supporting member.

As described above, there are a first pitch circle at which the driving wire W is provided and a second pitch circle which is obtained by offsetting the first pitch circle outside and at which the output shaft Ma is provided, and the tractor supporting shaft 21cs is provided along a third pitch circle which is obtained by offsetting the first pitch circle outside and offsetting the second pitch circle inside. Such a layout of the driving force transmission mechanism enables making the inter-axis distance Lt between the output shaft Ma and the tractor supporting shaft 21cs smaller and thus making the moment Mt applied to the tractor 21ct during driving smaller. With this configuration, it is possible to reduce a resistance acting on the tractor supporting shaft 21cs and thus reduce an abrasion between the output shaft Ma and the screw portion St of the tractor 21ct. Moreover, it is possible to make the difference Lr between radii of pitch circles obtained before and after changing formed by the driving wires W before and after bending smaller, make a moment for further bending the driving wire W during driving smaller, and prevent or reduce the occurrence of a frictional force in a direction to hinder the movement of the driving wire W. With this configuration, it is possible to reduce a loss of driving force transmission to the driving wire W. Accordingly, it is possible to provide a continuum robot capable of improving durability and implementing the smooth movement of the driving wire W.

Furthermore, in at least one embodiment, a configuration in which the catheter unit or portion 100 is attachable to and detachable from the base unit or portion 200 is employed. Specifically, a configuration described with reference to FIGS. 15A to 15C to FIGS. 17A to 17C is provided in such a way as to enable the user to readily replace the catheter unit or portion 100. As mentioned above, the plate spring 21ch serving as a holding portion and the held portion Wa are switched between a state in which they are fixed to each other and a state in which fixing of them has been released, so that the catheter unit or portion 100 is configured to be attachable to and detachable from the base unit or portion 200. Then, an interlocking portion (the joint 28 and the internal gear 29) which interlocks with the movement of the operation unit or portion 400 is used to perform switching between a state in which the plate spring 21ch and the held portion Wa are fixed to each other and a state in which fixing of the plate spring 21ch and the held portion Wa has been released. In this configuration, the interlocking portion is arranged on the outer side of a predetermined region of the coupling portion 21c, in at least one embodiment, on the outer side of a region closer to the catheter 11 than the output shaft Ma (see FIG. 8A). With this configuration, the interlocking portion is arranged with use of a space on the outer side of the first connection member or the tractor supporting shaft 21cs, which is arranged at a position offset inside in the radial direction of the pitch circle with respect to the output shaft Ma, so that it is possible to prevent or reduce an increase in size of the entire device. Moreover, it is possible to arrange the interlocking portion near the operation unit or portion 400 and thus reduce a force required for operating the operation unit or portion 400.

Furthermore, the advantageous effects of one or more features of the present disclosure may be attained without recourse to capability or incapability of the attachment and detachment of the catheter unit or portion 100 with respect to the base unit or portion 200. Thus, even in a configuration which does not include the configuration described with reference to FIGS. 15A to 15C to FIGS. 17A to 17C and in which replacement of the catheter 11 is unnecessary or simple replacement thereof by the user is unnecessary, employing the layout of a driving force transmission mechanism illustrated in FIG. 18 and FIGS. 19A and 19B enables improving durability and implementing the smooth movement of the driving wire W.

While the present disclosure has been described based on one or more embodiments thereof, the above-described one or more embodiments are merely examples of substantiation in implementing one or more features of the present disclosure, and the technical scope of the present disclosure should not be construed to be limited by such embodiment examples. Thus, one or more of the features of the present disclosure may be implemented in various manners without departing from the technical idea thereof or the principal features thereof.

According to aspects of the present disclosure, it is possible to provide a continuum robot capable of improving durability and implementing the smooth movement of a linear member.

While one or more features of the present disclosure have been described with reference to one or more embodiments, it is to be understood that the disclosure is not limited to the disclosed one or more embodiments. The scope of the following claims is to be accorded the broadest interpretation so as to encompass all such modifications and equivalent structures and functions.

This application claims the benefit of Japanese Patent Application No. 2021-202426 filed Dec. 14, 2021, which is hereby incorporated by reference herein in its entirety.

What is claimed is:

1. A continuum robot comprising:
a bendable body having a plurality of linear members provided along a first pitch circle, and a plurality of motors respectively having a plurality of output shafts provided along a second pitch circle which is offset outside from the first pitch circle and being configured to respectively drive the plurality of linear members to bend the bendable body;
a plurality of intermediate supporting shafts provided along a third pitch circle which is offset outside from the first pitch circle and is offset inside from the second pitch circle;
a plurality of first connection members respectively connecting end portions of the plurality of linear members and the plurality of intermediate supporting shafts to each other; and
a plurality of second connection members respectively connecting the plurality of intermediate supporting shafts and the plurality of output shafts to each other and configured to convert rotation of each of the plurality of output shafts into rectilinear motion to cause each of the plurality of intermediate supporting shafts to rectilinearly move.

2. The continuum robot according to claim 1, further comprising:
a first supporting member configured to support a respective one of the plurality of motors; and
a second supporting member configured to support a coupling portion including a respective one of the plurality of intermediate supporting shafts, a respective one of the plurality of first connection members, and a respective one of the plurality of second connection members,
wherein the first supporting member and the second supporting member are coupled to each other via a supporting portion coupling portion arranged inside the coupling portion.

3. The continuum robot according to claim 2, further comprising a positioning shaft arranged inside the coupling portion,
wherein the positioning shaft is coupled to the supporting portion coupling portion.

4. The continuum robot according to claim 1,
wherein a holding portion of each of the plurality of first connection members and a held portion of each of the plurality of linear members are configured to be attachable to and detachable from each other by being switched between a state in which the holding portion and the held portion have been fixed to each other and a state in which fixing of the holding portion and the held portion have been released,
wherein the holding portion and the held portion are switched by an interlocking portion which interlocks with movement of an operation unit or portion between the state in which the holding portion and the held portion have been fixed to each other and the state in which fixing of the holding portion and the held portion have been released, and
wherein the interlocking portion is arranged outside a predetermined region of a coupling portion including a respective one of the plurality of intermediate supporting shafts, a respective one of the plurality of first connection members, and a respective one of the plurality of second connection members.

5. The continuum robot according to claim 4, wherein the operation unit or portion is configured to be rotatable around a rotational axis extending in directions to attach and detach the held portion to and from the holding portion.

6. The continuum robot according to claim 1, further comprising a first bending portion and a second bending portion configured to bend a respective one of the plurality of linear members in such a way as to change a diameter of the first pitch circle,
wherein a difference between radii before and after changing of a pitch circle is smaller than a distance in an axial direction of the pitch circle between the first bending portion and the second bending portion.

7. The continuum robot according to claim 6, further comprising a pipe-like bending guide including the first bending portion and the second bending portion,
wherein a respective one of the plurality of linear members is configured to be movable inside the bending guide.
